(19) United States
(12) Patent Application Publication
(10) Pub. No.: US 2015/0183822 A1
(43) Pub. Date: Jul. 2, 2015

(54) ANGIOTENSIN-CONVERTING-ENZYME INHIBITING DIPEPTIDE

(71) Applicant: YAMAKI Co., Ltd., Iyo-shi, Ehime (JP)

(72) Inventors: Eiji Seki, Iyo-shi (JP); Hitoshi Asada, Iyo-shi (JP)

(21) Appl. No.: 14/411,454

(22) PCT Filed: Apr. 10, 2013
(86) PCT No.: PCT/JP2013/060847

§ 371 Date: Mar. 17, 2015

(30) Foreign Application Priority Data
Jun. 26, 2012 (JP) 2012-143061
Nov. 21, 2012 (JP) 2012-255001

(51) Int. Cl. C07K 5/078; A23L 1/305; C07K 5/06; C07K 5/072; A61K 38/05; C07K 5/062

(52) U.S. Cl. CPC C07K 5/06156; A61K 38/05; C07K 5/06043; C07K 5/06052; C07K 5/06; C07K 5/06069; C07K 5/06113; C07K 5/06026; A23L 1/3053; A23V 2002/00

(57) ABSTRACT

[Problem] To provide a useful dipeptide-containing composition derived from dried bonito flakes and the like, having an angiotensin-converting-enzyme inhibiting activity that imparts a blood pressure reducing function. [Solution] A composition containing a dipeptide derived from a fish meat protein having an angiotensin-converting-enzyme inhibiting activity. The composition is characterised by containing: at least one type of dipeptide selected from the group consisting of a dipeptide consisting of a tryptophan-leucine amino acid sequence, a dipeptide consisting of a leucine-tryptophan amino acid sequence, a dipeptide consisting of a tryptophan-isoleucine amino acid sequence, a dipeptide consisting of a valine-triptophan sequence, a dipeptide consisting of a tryptophan-tyrosine sequence, a dipeptide consisting of a tryptophan-methonine sequence, a dipeptide consisting of a serin-triptophan sequence, and a dipeptide consisting of an asparagine-triptophan sequence; and/or an acid addition salt of said dipeptide(s).

### TECHNICAL FIELD

The invention relates to useful 15 types of dipeptides demonstrating the anti-hypertensive action by virtue of the angiotensin converting enzyme (ACE) inhibitory activity thereof, and to a peptide composition comprising them. A method for producing the dipeptides and the dipeptide composition according to the invention is characterized by binding dipeptides to a hydrophobic resin, wherein the dipeptides are obtained by enzymatically hydrolyzing insoluble proteins as residues remained after hot-water treatment of a fish meat protein, especially a dried bonito (KATSUOBUSHI), with PROTIN NY100 enzyme (produced by AMANO ENZYME co.), and desorbing the dipeptides with alcohol including water, and thereafter, and then, passing the dipeptides through an ultrafiltration membrane (molecular weight: 1000), and the obtained fraction having high ACE inhibitory activity is beneficial as an angiotensin converting enzyme inhibitor and an active ingredient for anti-hypertensive agent. Dipeptides having high ACE inhibitory activity and peptide compositions comprising them would be expected to be beneficial for the treatment or prevention of hypertension.

### BACKGROUND OF THE ART

Hypertension is typical lifestyle-related disease, and the number of the hypertension patients and patients likely to hypertension in Japan is recognized to be 54,900,000 people (Health, Labor and Welfare Ministry: National Health and Nutrition Examination Survey, 2006). Further, it has been reported that people of one quarter of world population has hypertension or is likely to hypertension, in the survey by WHO of 2012. Hypertension is called Silent Killer because of the lack of the subjective symptom, is known to cause complicating disease such as cerebral hemorrhage, subarachnoid hemorrhage, cerebral infarction, myocardial infarction, angina pectoris, renal sclerosis and the like, and the various investigations have been conducted about the pathogenic mechanism of hypertension.

Renin-Angiotensin System relating to hypertension and Kallikrein-Kinin system relating to anti-hypertension play an important role as the control system for the blood pressure. In Renin-Angiotensin System, angiotensinogen is converted to angiotensin I with renin produced in kidney, and further to angiotensin II with angiotensin converting enzyme (ACE). The angiotensin II contracts the vascular smooth muscle, and whereby, increases the blood pressure. On the other hand, the anti-hypertension system, kallilrein, acts on kininogen to produce bradykinin. Although bradykinin has effects to extend the blood vessel to decrease the blood pressure, ACE has the action to decompose bradykinin. Thus, it has been known that ACE relates the increase of the blood pressure due to the production of a hypertension peptide, angiotensin II, and the inactivation of anti-hypertension peptide, bradykinin.

It becomes possible, therefore, to prevent the increase of the blood pressure by preventing the enzymatic activity of ACE. Proline derivatives such as captopril (D-2-methyl-3-mercaptopropanoyl-L-proline) or enalapril developed as an ACE inhibitory active material has been widely used for treatment of the hypertension. In the pharmaceutical, however, the side effect, the dry cough is recognized by dose, and hence, it is also the fact that there is a problem in the aspect of QOL. The rebound is also known after the cessation of the drug.

Thus, it tends to search or purify to utilize a component having the anti-hypertensive action from a natural product. Amongst them, recently, it is reported that a peptide being enzymatic decomposition product from a food material protein has the ACE inhibitory activity. It is reported numerously, for example, a gelatin decomposition product with collagenase (Patent document 1), a casein decomposition product with trypsin (Patent documents 2, 3 and 4), WASHI muscle decomposition product with pepsin (Patent document 5), KATSUOBUSHI decomposition product with thermolysin (Patent document 6), sesame protein decomposition product with thermolysin (Patent document 7), κ-casein decomposition product with pepsin (Patent document 8) and the like. Because these peptides, namely, angiotensin converting enzyme inhibitor derived from a natural product can be obtained from a food or a food material, these peptides are expected to be a low toxicity and a high-safety anti-hypertensive agent.

It has been found out ACE inhibitory substance in microorganism or various foods, and considered the practical realization as an anti-hypertensive agent (Non-patent document 1).

In addition, it have been reported several methods for producing a peptide having the ACE inhibitory activity (Patent documents 9, 10, 11, 12, 13, 14 and 15).

### PRIOR ART DOCUMENTS

### Patent Documents

Patent document 1: JP Kokai Sho52-148631
Patent document 2: JP Kokai Sho58-109425
Patent document 3: JP Kokai Sho61-36226
Patent document 4: JP Kokai Sho61-36227
Patent document 5: JP Kokai Hei3-11097
Patent document 6: JP Kokai Hei4-144696
Patent document 7: JP Kokai Hei8-231588
Patent document 8: JP Kokai Hei8-269088
Patent document 9: JP Kokai Hei6-7188
Patent document 10: JP Patent 2794094
Patent document 11: JP Patent 2873318
Patent document 12: JP Kokai 2006-347937
Patent document 13: JP Kokai 2001-240600
Patent document 14: JP Kokai Hei6-298794
Patent document 15: JP Kokai 2010-155788

### Non-Patent Documents

K. Suetsuna, “Fermentation and Industry”, Vol. 46 (No. 3), pp 179-182, 1988

### SUMMARY OF THE INVENTION

### Problems to be Resolved by the Invention

A peptide having the ACE inhibitory activity which is derived from a food material is markedly advantageous to its little problem in the safety such as the side effect and the toxicity and the possibility for the ingestion as a normal food.

Whereas, almost of peptides as reported as above are these peptides having the constituting amino acid number of 5 or more (Patent document 1, 2, 3, 4, 5 and 8). It is recognized that such the peptide having the many amino acid residues are easy to be decomposed with a digestive enzyme such as pepsin, trypsin, chymotrypsin and the like after the ingestion, the ACE inhibitory activity of such the peptide would be lost in vivo, and if not decomposed, such the peptide is hard to be absorbed due to its large molecular structure.

Regarding an ACE inhibitory peptide composition and a method for producing thereof, ACE inhibitory peptide mixture having the blood pressure lowing action was obtained in a fraction which a low molecular component was removed, from liquid passing through an ultrafiltration membrane (M.W.3000-10000), with a reverse osmotic membrane in Patent document 9, although the document intends to increase the inhibitory activity by removing salts and free amino acids, does not intend to fractionate, and not intend to clarify the tracking of the concrete component peptide with the reverse osmotic membrane. Although Patent document 10 discloses that an aimed oligopeptide can be obtained from a substance which has the molecular weight of 10000 or less and is derived from fig tree by isolating and purifying with a column chromatography, the disclosure of the document remains the productivity by synthesis and the isolating method, and does not disclose that the peptide can be effectively and industrially produced from a natural product concretely, and further, show nothing but the purification with an ultrafiltration membrane of 10000. Although Patent document 11 discloses a method for obtaining a zein or a gluten meal hydrolyzate with thermolysin which the content of a fraction having molecular weight of 10000 or less is 30% or more based on solid content by use of the gel filtration or the ultrafiltration, the document does not demonstrate the basis that the hydrolyzate content having molecular weight of 1000 or less which is contained in permeable liquid from a membrane of M.W.10000, is 95%. Whereas it is obtained a composition which includes an anti-hypertensive peptide which is derived from a livestock meat protein and which myosin and actin are hydrolyzed with a enzyme such as AMANO S in Patent document 12, the purification remains lab-scale, and the synthesis of an isolated peptide is described. The industrial bulk production in the document is only described as a general production by using an enzyme. In addition, a resin is used for the purification of the inhibitory peptide in order to determine the structure of the peptide. Patent document 13 discloses a method for treating a hydrolyzate of a protein, liquid including ACE inhibitory peptide, with an activated carbon having an average pore diameter of 3 nm or less, and that it was possible to remove the bitterness and the smell in the liquid without the decrease of ACE inhibitory activity. However, the document is not intended to increase the inhibitory activity. Patent document 14 proposes a method for contacting a hydrolyzate of a protein with a synthetic resin to remove a bitter peptide into an unbound fraction, and describes that the inhibitory activity remains in the unbound fraction. On the one hand, the high ACE inhibitory activity can be recognized in a bound fraction, and the ACE inhibitory activity was not recognized in an unbound fraction, and a low molecular ACE inhibitory active peptide which is advantageous to the digestion resistance can be obtained in a permeable liquid from an ultra filtration, in the invention of this application.

The obtained peptide having the ACE inhibitory activity can be purified, isolated and characterized as a dipeptide having a high inhibitory activity by subjecting the peptide to HPLC, at one operation. This supports that the ACE inhibitory peptide defined in the invention of this application can be fractionated with high accuracy, and suggests that unnecessary peptide is low, contrary to the indication that the isolation of a peptide from an enzymatic decomposition product requires a several or five times column operations. Further, the ACE inhibitory peptide defined in the invention of this application demonstrates the high titer of anti-hypertensive effect because the peptide is a collectivity of peptides having the high ACE inhibitory activity.

A problem to be resolved of the invention of this application is, thus, to provide a dipeptide having the ACE inhibitory activity which is hard to be decomposed with a digestive enzyme when it is ingested orally, which the ACE inhibitory activity thereof is hard to be lost in the body, and which can be absorbed into a mucosa of a small intestine, on its own, and a peptide composition comprising the dipeptide.

Moreover, the invention of this application provides an angiotensin converting enzyme inhibitory agent, a blood pressure lowering agent or a food and beverage composition (a food and beverage or a food and beverage for specified health use) comprising at least one said peptide.

As described above, it is very important an angiotensin converting enzyme inhibitory substance derived from a natural organic substance and a food since it is safe to the human body, and therefore, it is a large research problem to prevent a lifestyle-related disease.

The invention of this application was completed in order to resolve the problem, and the subject of the invention was to find a novel and safe material which prevents the increase of the blood pressure by inhibiting angiotensin converting enzyme from a material food, to clear the structure of the inhibitory material, to develop the proper method for condensing the material at the point of the quality and the costs, and further, to provide a food material comprising the angiotensin converting enzyme inhibitory substance.

### Means of Solving the Problems

The inventors consider that the peptide able to resolve the problem described as above exists in the decomposition product which is obtained by hydrolyzing a water-insoluble protein with a protease, PROTIN NY100 (AMANO ENZYME CO., Ltd.), wherein the protein remains as a residue after the extract of KATSUOBUSHI with hot water, and explores whether a peptide having the amino acid number of 2 or less and ACE inhibitory activity exist or not in the decomposition product.

In consequence, the inventors found out five dipeptides having the following amino acid sequences and the ACE inhibitory activity in a product obtained by the hydrolysis of the water-insoluble protein, the residue remained after extracting KATSUOBUSHI with the hot water, succeeded in the isolation of these dipeptides, and thus, completed the invention of this application.

The invention of this application provides a composition characterized by being derived from a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and comprising dipeptide having the angiotensin converting enzyme inhibitory activity, wherein the dipeptide is at least one dipeptide selected from a group consisting of

a dipeptide composed of a tryptophan-leucine amino acid sequence,

a dipeptide composed of a leucine-tryptophan amino acid sequence,

a dipeptide composed of a tryptophan-isoleucine amino acid sequence,

a dipeptide composed of a valine-tyrosine amino acid sequence,

a dipeptide composed of a tryptophan-asparagine amino acid sequence,

a dipeptide composed of a valine-tryptophan amino acid sequence,

a dipeptide composed of a tryptophan-tyrosine amino acid sequence,

a dipeptide composed of a tryptophan-methionine amino acid sequence,

a dipeptide composed of a methionine-tryptophan amino acid sequence,

a dipeptide composed of a isoleucine-tryptophan amino acid sequence,

a dipeptide composed of a serine-tryptophan amino acid sequence,

a dipeptide composed of an asparagine-tryptophan amino acid sequence,

a dipeptide composed of a glutamine-tryptophan amino acid sequence,

a dipeptide composed of a glycine-tryptophan amino acid sequence and

a dipeptide composed of an alanine-tryptophan amino acid sequence, and/or acid addition salts thereof.

The invention of this application, further, relates to a composition, characterized in that the composition is derived from a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence and a dipeptide composed of a tryptophan-asparagine amino acid sequence.

The invention of this application, further, relates to a composition, characterized in that the composition is derived from a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and comprises a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence and a dipeptide composed of an isoleucine-tryptophan sequence.

The invention of this application, further, relates to a composition, characterized in that the composition is derived from a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and comprises a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

The invention of this application, further, relates to a composition, characterized in that the composition is derived from a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence, and/or acid addition salts thereof.

The invention of this application, further, relates to a processed food or a food for specified health use, characterized by comprising any one of compositions described above.

The invention of this application, further, relates to a pharmaceutical composition, for example, an antihypertensive composition, characterized by comprising any one of compositions described above.

Moreover, the invention of this application relates to a method for producing a composition comprising at least one dipeptide selected from a group consisting of a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence, and/or acid addition salts thereof, characterized in that the method consists of:

1) extracting a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish with hot water,

2) grinding a water-insoluble protein remained after the hot water-extraction, and reacting, the water-insoluble protein particle obtained by dispersing the obtained ground product into water, with protease under the optimum condition of pH5.0 to pH9.0 at the temperature of 40 to 60° C., and thereby, enzymatically hydrolyzing the water-insoluble protein, and then, stopping the enzyme reaction, and removing water-insoluble particles from the obtained hydrolysis reaction mixture including water, and thereby, obtaining aqueous solution comprising hydrophobic and hydrophilic high molecular and low molecular peptide as well as a water-soluble amino acid, and

3) loading and passing a bound fraction obtained with hydrophobic resin column method to ultrafiltration (molecular weight: 1000) to finally purify the fraction.

Moreover, the invention of this application relates to a composition, characterized in that the composition is obtained according to the said method and comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence and a dipeptide composed of a tryptophan-asparagine amino acid sequence.

The invention of this application, in addition, relates to a composition, characterized in that the composition is obtained according to the said method and comprises a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence and a dipeptide composed of an isoleucine-tryptophan sequence.

The invention of this application, in addition, relates to a composition, characterized in that the composition is obtained according to the said method and comprises a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

The invention of this application, in addition, relates to a composition, characterized in that the composition is obtained according to the said method and comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

Further, the invention of this application relates to a processed food, a food for specified health use, or a pharmaceutical composition, for example, an antihypertensive composition, characterized by comprising the composition obtained according to the said method.

The inventors had been variously investigated, and as the result, could presumed that an angiotensin converting enzyme inhibitory substance exists in a bonito protein, and clarified that the angiotensin converting enzyme inhibitory substance in the bonito protein has the property that it had been bound to a reversed-phase partition resin. Also, it could be clarified that a high activity fraction having the digestion resistance could be obtained in the permeable liquid from an ultrafiltration (M.W.1000) membrane. It could be clarified, in addition, that the inhibitory substance could be obtained in ACE inhibitory fraction in the high yield, by decomposing a insoluble protein residue produced by hot water extraction of KATSUOBUSHI material with a protease, preferably, the protease for the food industry use, more especially, PROTIN NY100 (AMANO ENZYME CO., Ltd.), binding the decomposed substance to a hydrophobic absorptive resin, eluting it with organic solvent including water, and subjecting it to the ultrafiltration membrane treatment (M.W. 1000), as well as could be concentrated easily, and further, that a component having the high ACE inhibitory activity amongst the each component in the resulting inhibitory substances was isolated with UPLC chromatography and the measurement of the inhibitory activity value (IC₅₀ value) and the structure of the components were analyzed, and as the result, the components were the dipeptides having the amino acid sequences described above and the angiotensin converting enzyme inhibitory activity.

The invention of this application, as means for resolving the object described above, provides the angiotensin converting enzyme inhibitory substance which mainly comprises the angiotensin converting enzyme inhibitory peptide selected from a group consisting of a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence, and further, the angiotensin converting enzyme inhibitory substance characterized by existing in the permeable liquid in the high ACE inhibitory activity which is obtained by decomposing a insoluble protein residue produced by hot water extraction of KATSUOBUSHI with a protease, for example, PROTIN NY100 (AMANO ENZYME CO., Ltd.), thereafter, binding the decomposed substance to a hydrophobic absorptive resin, eluting it with organic solvent including water, and subjecting it to the ultrafiltration membrane treatment (M.W.1000). Because the ultrafiltration membrane treatment requires generally several days, there is no example for the use of the treatment in the food, until now, since the problem of the composition carrion has not been resolved. Lower temperature or lower pH, as means for resolving the problem, causes the problems of the facility cost, the running cost and the quality due to the use of hydrochloric acid, and hence, these cannot be a clear solution approach. The use of the ultrafiltration membrane (M.W.10000) together with the reverse osmotic membrane cannot exactly conduct the molecular weight fraction. As the result of the improvement of these problems, the invention of this application provides ACE inhibitory peptide obtained by the production process which the desorption of the hydrophobic resin treatment is conducted by use of 50% ethanol solution to remove the inhibitory inactive fraction and the free amino acid, and thereby the ACE inhibitory activity is increased in the first purification step, and then, the high active component can be recognized in the permeable liquid obtained by the ultrafiltration membrane (M.W.1000) treatment process under the presence of alcohol, and which does not cause the carrion and stable.

A hydrophobic adsorptive resin, namely, an modified aromatic type resin (for example, Sepabeads SP207; produced by MITSUBISHI CHEMICAL CORP.) is an aromatic based on brominated aromatic matrix (styrene-divinylbenzene) synthetic adsorbent, it is recognized that the resin provides excellent adsorbing performance to the high hydrophilic organics (the low hydrophobic substance), as well, because the hydrophobic adsorbability the pore surface is strong, and therefore, the resin may be used in the amino acid separation and purification, the protein removal, the natural extract purification, the pre-treatment with the fermentation liquor and the like.

Dipeptides used in the invention of this application can be produced by use of a method for enzymatically decomposing a hot water extraction residue protein produced from KATSUOBUSHI, a method for introducing gradually an amino acid by the organic chemical synthesis method, the peptide synthesis method using the reverse reaction of the hydrolase, the genetic engineering method and the like.

We would explain, hereinafter, the said method for producing the peptide by use of the method for enzymatically decomposing the water-insoluble protein, namely, the hot water extraction residue produced from KATSUOBUSHI, and further, the case that more purified-hot water extracted-insoluble fraction is used as the KATSUOBUSHI protein material.

The protease for the food industry use can be preferably exemplified as an action enzyme. PROTIN NY100 (AMANO ENZYME CO., Ltd.), for example, can be exemplified. PROTIN NY100 (AMANO ENZYME CO., Ltd.) is derived from Bacillus amyloliquefaciens, the optimum pH of 7.0 and the optimum temperature of 55° C. On one hand, the substrate concentration may be in the range such that it can be stirred and admixed, preferably, in the range of the protein concentration of 2 to 30% (w/v) which is easy to be stirred. The additive amount of the enzyme is varies according to titer of the protease, properly 0.01 wt. % or more, preferably 0.1 to 10 wt. %, normally, based on the protein. The pH and the temperature for the reaction may be around the optimum pH and the optimum temperature, for example, 5.0 to 9.0, preferably 5.0 to 7.5 of pH, and 40 to 60° C., preferably 45 to 55° C. of the temperature. The pH during the reaction is, as necessary, adjusted with sodium hydroxide solution or hydrochloric acid.

The enzyme reaction time varies and is not set because of dependent on the addition amount of the enzyme, the reaction temperature and the reaction pH, but normally, about 1 to 50 hours.

The termination of the enzymatically decomposition reaction can be made by means of the publicly-known method such as the deactivation of the enzyme by the heating of the hydrolyzate or pH alteration. Then, the hydrolyzate liquid is separated to the solid and the liquid (such as the centrifugation or the filtration), and the separated liquid is separated with the ultrafiltration or the gel filtration to obtain the liquid including the fraction of, for example, M.W.10000 or less. The liquid or concentrate thereof (for example, spray dry) including the dipeptide defined in the invention of this application is further successfully separated to obtain the composition comprising the target dipeptide.

The acid addition salt of the dipeptide can be produced with the ordinary method. For example, the acid addition salt can be obtained by reacting and lyophilizing the dipeptide of the invention (including the basic amino acid residue) and one equivalent of the proper acid based on the dipeptide in the water.

The composition comprising the dipeptide of the invention or the acid addition salt thereof has the ACE inhibitory action, eventually the anti-hypertensive action, and is whereby expected to be effective in the treatment and the prevention of the hypertension of the mammal including the human.

The composition comprising the dipeptide of the invention or the acid addition salt thereof is used in situ, or generally, as a pharmaceutical composition combined it with a pharmaceutical adjuvant.

The composition comprising the dipeptide of the invention or the acid addition salt thereof can be formulated to appropriate form to various administration method such as parenteral (i.e. intravenous injection or rectal administration) or oral administration.

The formulation as an injectable solution, generally, comprises the sterilized aqueous solution. The said formulation can comprise further the other pharmaceutical adjuvant than the water such as buffer, pH adjuster (sodium hydrogenphosphate, citric acid, etc.), tonicity agent (sodium chloride, glucose, etc.), preservative (methyl parahydroxy benzoate, propyl p-hydroxybezoate, etc.). The formulation can be sterilized with the filtration passing through the filter for holding virus, the introduction of disinfectant into the composition, or irradiation or heating of the composition. The formulation also can be produced as a sterilized solid composition, and used by dissolving it with sterilized water in use.

The orally-administered agent is formulated to the appropriate form in gastrointestinal absorption. Tablet, encapsulated formulation, granule formulation, fine granule formulation and powder formulation can includes the conventional pharmaceutical adjuvant, for example, the binder (syrup, gum Arabic, gelatin, sorbitol, gum tragacanth, polyvinylpyrrolidone, hydroxypropylcellulose, and the like), the excipient (lactose, sugar, cornstarch, calcium phosphate, sorbitol, glycine and the like), the lubricant (magnesium stearate, talc, polyethyleneglycol, silica, and the like), the disintegrant (potato starch, carboxymethyl cellulose and the like) and the wetting agent (sodium lauryl sulfate and the like). The tablet can be coated by use of the ordinary method. The oral solution can be formed to the aqueous solution, the dry product. Such the oral solution may comprise in common use additive such as the preservative (methyl- or propyl-p-hydroxy benzoate, sorbic acid and the like).

The amount of the composition comprising the ACE inhibitor or the amount of the composition comprising the dipeptide of the invention or the acid addition salt thereof in the anti-hypertensive agent, can be varied, and is generally 5 to 10% (w/w), and especially 10 to 60% (w/w). The dose of the ACE inhibitor or the anti-hypertensive agent is, if it is administered to the human, properly 0.01 to 50 mg/kg/day as an active ingredient.

The composition, in addition, can be eaten in situ or as a functional food or a health food which has the anti-hypertensive action or the prevention to the hypertension, by adding additionally the various nutritive substance to them, or by involving them into the beverage and food because the composition comprising the dipeptide of the invention has the advantage that it does not negatively affect to the biological body even if they are eaten in sufficient quantity. That is to say, the composition of the invention can be used, for example, as the liquid food such as an energy drink, soy milk, soup and the like, or the various form of the solid food by adding the nutritive substance such as a variety of vitamins, minerals and the like, and further, as the powder in situ or as the state of the additive into a variety of the foods. The content and the dose of the active ingredient in the ACE inhibitor or the anti-hypertensive agent of the invention as the functional food or the health food are the same content as the content and the dose for the pharmaceutical formulation as described above.

The organic chemical synthesis methods of said dipeptide are two methods of the liquid phase- and the solid phase method, which can be done according to ordinary methods such as “The base and the experiment of peptide synthesis”, N. IZUMIYA, T. TETSUO, A. AOYAGI and M. WAKI, MARUZEN CO., Ltd., 1985. In the liquid phase method, amino acid which should locate on the C-terminal of the peptide of the invention and whose carboxyl group is protected with the benzyl group (Bzl), t-butyl group (t-Bu) and the like, and the amino acid which should locate on the next to said C-terminal amino acid and whose α-amino group is protected with t-butyloxycarbonyl group (Boc), benzyloxycarbonyl group (Z) and the like, are dissolved in dimethylformamide (DMF), dimethylacetamide and the like, and further, these amino acids are reacted in the presence of dicyclohexylcarbodiimide (DCC) and 1-hydroxy benzotriazole (HOBT) at the ambient temperature for overnight. Then, the dipeptide derivative which the amino protective group of the product is removed according to the ordinary method is, if necessary, reacted with the third amino acid whose amino group is protected, similarly, the amino protective group is removed, if necessary, the identical procedures are repeated, and thereby, the dipeptide derivative of the invention is obtained. In the case that reacting amino acid has hydroxyl group, guanidino group or imidazolyl group, these group should be generally protected prior to said reaction. The protective group of alcoholic hydroxyl group includes Bzl, t-Bu and the like, the protective group of phenolic hydroxyl group includes Bzl and the like, the protective group of guanidino group includes tosyl group (Tos) and the like, and the protective group of imidazolyl group includes Tos. After the termination of the final reaction, all the protective groups are removed to obtain the dipeptide of the invention. These protective groups are introduced and removed in the ordinary method.

On the other hand, regarding the solid phase method, the peptide synthesizer, recently, have been widely used, the dipeptide of the inventions can be produced by use of 430A-type Peptide Synthesizer (made by Applied Biosystems CO., Ltd.), for example. That is to say, basically, α-amino acid which the amino group is protected with Boc (Boc-amino acid) is gradually extended by the repeat of the binding of peptide and the removal of Boc, from the N-terminal side of phenylacetamidemethyl (PAM) resin, L-Xaa-O—CH₂—PAM, wherein Xaa is amino acid residue, available from Applied Biosystems CO., Ltd., to which amino acid located on C-terminal of the dipeptide of the invention binds. Boc-amino acid is subjected to the extension reaction by use of DCC via the symmetric anhydride as an intermediate thereof. If there is the reactive functional group which should not involve with the reaction in Boc-amino acid or L-Xaa-O—CH₂—PAM, the functional group should be generally protected with the proper protective group. In the synthesis system using 430A-type Peptide Synthesizer, the following reagent and solvent in addition to the amino acid material: N,N-diisopropylethylamine (TFA neutralizer), TFA (Boc cleavage), MeOH (the dissolution and the removal of produced urea compound), HOBT (0.5M HOBT/DMF), DCC (0.5M DCC/dichloromethane(DCM)), DCM and DMF (the solvent), the neutralizer (70% ethanol amine, 29.5% methanol)(the neutralization of waste liquid). Amino acid material, these reagents and the solvents are loaded on the predefined location. The peptide synthesizer automatically uses them. The adjustment of the reaction temperature and time is also done automatically, wherein the reaction temperature is normally room temperature. The dipeptide-O—CH₂—PAM which the reactive group in the dipeptide is protected according to the abovementioned procedure, can be obtained. The actual operation for the solid phase peptide synthesis as described above is done in accordance with the user manual for 430A-type Peptide Synthesizer, provided by Applied Biosystems CO., Ltd.

The intended dipeptide is obtained by treating the obtained dipeptide-O—CH₂—PAM which the reactive functional group is protected, according to an ordinary method, for instance, a method described in the said “The base and the experiment of peptide synthesis” or the user manual for 430A-type Peptide Synthesizer, for instance, a method for treating the dipeptide with trifluoromethanesulfonate (TFMSA) together with TFA (TFA is diluent for TFMSA) under the presence of thioanisole and/or ethanedithiol as the scavenger which captures a cation produced by cleave of the protective group to cleavage the resin and the protective group.

The dipeptide of the invention may be produced by the organic synthesis described as above. However, for the purpose of adding it to the beverage and food or the pharmaceuticals and providing the ACE inhibitory activity, it is preferable to produce the dipeptide as the oral ingestible composition comprising at least one of 15 dipeptides described above by decomposing the protein derived from KATSUOBUSHI and the like with PROTIN NY100 (produced by AMANO ENZYME Co., Ltd.), and subsequently isolating and purifying it.

These can be used as the material a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and the hot water extraction residue of them.

In the case of obtaining the dipeptide of the invention by decomposition with PROTIN NY100 (produced by AMANO ENZYME Co., Ltd.), it is preferable, as a pretreatment, to remove amino acids and water-soluble proteins produced in the heating treatment. Further, it is preferable to stir the material and suspend it in water after it is ground finely in order to increase the efficiency of the enzymatic decomposition by PROTIN NY100 (produced by AMANO ENZYME Co., Ltd.). Because the obtained protein is hard soluble, sodium hydroxide is added to it so that pH becomes to optimum one for the enzymatic reaction, and then, is uniformly dispersed, suspended and dissolved. Additionally, 0.1 to 10 wt. % of PROTIN NY100 (produced by AMANO ENZYME Co., Ltd.) on the basis of 100 g of protein is added to the solution, the protein decomposition is conducted at pH5.0 to 9.0 at the temperature of 40 to 55° C. for 0.5 to 30 hours with the stirring operation, and subsequently, the enzymatic activity is deactivated by the heating treatment (98° C., 15 minutes). Not decomposed protein in the decomposition liquid is removed with the vibrating screen, and then, the not decomposed material and the precipitation with the decanter, the deraval, the ultrafast centrifuge (1500 rotations/minute) or the filtration (the celite filtration: Hyflo Super Celite and the like), and the obtained filtrate is neutralized with sodium hydroxide or hydrochloric acid, and then, is condensed. The obtained KATSUOBUSHI peptide thus comprises 0.0005 wt. % to 0.5 wt. % of a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence, respectively.

The composition comprising dipeptide of the invention is the obtained enzymatic decomposition product with PROTIN NY100 (produced by AMANO ENZYME Co., Ltd.) described above, or the crude purified product abundantly comprising peptide of the invention can be obtained by further treating the decomposition product with high-porous polymer resin (the hydrophobic adsorptive resin) or the ion exchange resin to remove the high molecular protein, the monomer amino acid and the salts and passing to the ultrafiltration to remove the high-molecular peptide, and said the crude purified product can be used in situ. These decomposition product and the crude purified product are called as composition comprising abundantly dipeptide.

In the case that the composition comprising the dipeptide of the invention by the purification, the peptide fraction of the invention having the ACE inhibitory activity is collected with the gel filtration chromatography, the chromatography using the ion exchange resin or the high porous polymer resin, the affinity chromatography and the like, and then, the active fraction can be purified to nearly pure each peptide by use of the general peptide purification method using the high-performance liquid chromatography with the reversed-phase column such as ODS column and the like. The dipeptide of the invention can be obtained from a bonito, the hot water-extraction residue derived from a bonito, a frigate mackerel, a dried frigate mackerel, a frigate mackerel, the hot water-extraction residue derived from a frigate mackerel, and the fish meat protein, by use of the methods shown as above. The ACE inhibitory activity of the dipeptide or the composition comprising abundantly it can be, for example, measured by use of the method described in Test Example 1, for instance.

In the case of obtaining the peptide of the invention, the peptide can be synthesized in any of the solid- or the liquid-phase method which is used in the general peptide synthesis. The peptide of the invention obtained by the synthesis can be purified by use of the general method using the reversed-phase high-performance liquid chromatography, the chromatography with the ion exchange resin or the high porous polymer resin, the affinity chromatography and the like. Then, the peptide can be obtained which has the increased ACE inhibitory activity and the digestion resistance by removing the high molecular peptide, with the ultrafiltration.

Thus obtained dipeptide or the composition comprising it numerously can be used as a remarkably available ACE inhibitory agent because the relative activity for the ACE inhibitory activity of it is high. The peptide and the composition comprising it can be applied to any of the form in the various beverage and food and the pharmaceutical formulation because the peptide and the composition are absorbed well from the intestine and relatively stable against the heat.

The invention of this application provides the drink and food composition (the drink and food) which at least one or more of the peptides or the compositions including them described above are incorporated and can exercise the angiotensin converting enzyme inhibitory action. The invention provides further the angiotensin converting enzyme inhibitory agent and the anti-hypertensive agent which comprises one or more of dipeptides described above.

In the case that the composition comprising the dipeptide of the invention is incorporated into food and beverage, and pharmaceuticals or used as it, it may be used the dipeptide which is sufficiently purified and derived from the product obtained by decomposition of the residue protein remained after extracting KATSUOBUSHI with the hot water by use of PROTIN NY100 (produced by AMANO ENZYME Co., Ltd.) enzyme, or the synthetic material obtained by the chemical synthesis. Because the peptide of the invention is stable and strong in the ACE inhibitory activity, the sufficient ACE inhibitory activity can be obtained by using the crude purified product or the enzymatic decomposition product using PROTIN NY100 (produced by AMANO ENZYME Co., Ltd.) in situ as the composition comprising numerously the dipeptide.

The beverage and food composition of the invention of this application (the beverage and the food) is produced by adding the composition comprising one or more of dipeptides as described, such that the amount of the dipeptide is 0.001 mg to 100 mg, and preferably 0.01 mg to 20 mg per a dose. The peptide composition of the invention of this application is the solid or the powder which is easy to use and stable, and is highly soluble to water. The composition, further, can be easy absorbed from the gastrointestinal tract. The time and the method for addition of the composition into the foods, therefore, is not particularly limited, and the composition can be added in the step as the material, in the intermediate process and in the final process of the food production, as the powder, the liquid or the suspension and the like, by use of the conventional method in the foods field. It is possible to inhibit the angiotensin converting enzyme, for example, to decrease the blood pressure by temporarily, intermittently, continuously and routinely consuming the beverage and food composition comprising the dipeptide of the invention. The solid, the semifluid or the fluid is provided as the form of the beverage and food. The general food and the health food in the form of the pill as the sheet, the tablet or the capsule and the like, or the granular powder, are provided as the solid food. It can be provided the general food and the health food in the form as the paste, the jelly or the gel, as the semifluid food, and the juice, the cold beverage, the tea or the drinkable preparation, as the fluid food. It is also possible to inhibit the increase of the blood pressure by consuming continuously the dipeptide of the invention as the energy drink or the seasoning.

The pharmaceutical composition in the form of the ACE inhibitory agent or the anti-hypertensive agent according to the invention of this application includes the composition which comprises the dipeptide of the invention in the same amount as the beverage and food composition as described above. The pharmaceutical composition of the invention may be temporarily administered to the anti-hypertensive patient in order to inhibit the angiotensin converting enzyme in the patient, for example, to exercise the decreasing action of the blood pressure, or can be continuously used safely because the active ingredient of pharmaceutical composition of the invention is derived from the natural material. The hypertension can be treated or prevented by use of the pharmaceutical composition of the invention. The forms of the pharmaceutical composition are preferably the orally-administered agent such as the tablet, the capsule, the granule, the syrup and the like. The sterile liquid formulation can be exemplified as the parenteral administration formulation because it is administered through the vein, the artery, the skin or the muscle, or inhaled from the nasal cavity. The liquid formulation may be the dried solid which is soluble in use. The injectable formulation can be produced by solving the dipeptide as the active ingredient into the saline and by use of the normal aseptic operation.

KATSUO-BUSHI as the law material is used, the amino acids and the water-soluble protein is removed with the hot water-extraction treatment for the material, and then, the insoluble protein residue is obtained, in the means defined in the invention of this application. The residue protein remained after hot-water extraction of KATSUOBUSHI is enzymatically decomposed and treated with PROTIN NY100 (AMANO ENZYME Co., Ltd.) enzyme.

The liquid after the enzymatically decomposition, subsequently, is treated with the vibrating screen, the deraval, the sharpless, or the celite filtration which is available in the effectiveness of the adsorption ability, and then, loaded on the column filled with the hydrophobic adsorptive resin, and adsorbed to the resin by flowing through the column.

The angiotensin converting enzyme inhibitory material which is adsorbed to the hydrophobic adsorptive resin, is eluted with the organic solvent containing water such as ethanol including water.

When the elution is conducted with the solvent, it is advantageous to treat by the supply of water for elution of the water-soluble substance before the supply of the solvent in order to effectively elute the adsorbed inhibitory substance as described above with the solvent. That is to say, the aqueous solution after the enzymatically decomposition has preferably the volumes of about twofold to tenfold to the column, and is loaded to the column, and subsequently, the volumes of about twofold to tenfold of water to the column is passed through. All of the unbound fractions are eluted. The desorption is conducted with ethanol of the concentration of 50% to obtain the objective bound fraction.

The fraction comprising the angiotensin converting enzyme inhibitory substance, which is eluted with ethanol solution, is loaded to the ultrafiltration (M.W.1000), the high-inhibitory active permeable liquid fraction is concentrated under the reduced pressure, and subsequently, is spray-dried to obtain the food material product as the powder formulation including mainly the angiotensin converting enzyme inhibitory peptide.

The food material including mainly the angiotensin converting enzyme inhibitory peptide also can be obtained as the purified and isolated form which has high angiotensin converting enzyme inhibitory activity, by isolating the component of the said eluted solution with the high-performance liquid chromatography and isocratic-eluting the component with acetonitrile/trifluoroacetic acid.

### The Advantageous Effect of the Invention

According to the invention, the fifteen dipeptides having the angiotensin converting enzyme inhibitory activity and the composition comprising at least one of them could be obtained by reacting KATSUOBUSHI as the material, which is the food and which the safety has been demonstrated on the basis of the long intake experience, with PROTIN NY100 (AMANO ENZYME Co., Ltd.). In addition, it was clear that the high-activity peptide fraction can be produced by binding the enzymatic decomposition to the hydrophobic adsorptive resin and eluting it with the organic solvent including water. Subsequently, it was also clear that the peptide having the high-ACE inhibitory and the digestion resistance could be obtained by passing the eluted solution through the ultrafiltration (M.W.1000) and recycling it. It was clear that the dipeptide demonstrates the anti-hypertensive action even if the dipeptide is small amount, in the animal testing. It is obvious that KATSUOBUSHI peptide comprising the dipeptide obtained according to the production method of the invention is the safe and available material as a food which is routinely consumed, and KATSUOBUSHI peptide is the significant food material for the future aging society and is expected to be utilized to the specified health food or the functional food. The production method of the invention can be widely utilized in the production of the functional material in the factory scale.

### BRIEF DESCRIPTION OF THE DRAWINGS

FIG. 1 is a graph which demonstrates the blood pressure lowing action to SHR for the peptide composition defined in the invention of this application in Example 7.

FIG. 2 is a graph which the graph shown in FIG. 1 is converted in %.

FIG. 3 is a graph which demonstrates the systolic blood pressure lowing action to SHR for the peptide composition defined in the invention of this application in Example 7.

FIG. 4 is a graph which demonstrates the systolic blood pressure lowing action to SHR, as the heart rate for the peptide composition defined in the invention of this application in Example 7.

### MODE FOR CARRYING OUT THE INVENTION

### Test Example 1

ACE inhibitory activity was measured as below. That is to say, ACE inhibitory activity of the peptide and the composition comprising it which was obtained as above, was measured according to Cheung and Cushman's method (Biochemical Pharmacology, 20, 1637 (1971)), except for substituting borate buffer for phosphate buffer.

5 g of rabbit lung acetone powder was dissolved in 50 mL of 0.1M sodium borate buffer (pH8.3), centrifuged under the condition of 40000G and for 40 minutes and resulting supernatant was purified with hydroxyapatite to obtain 1 unit/mg protein of angiotensin converting enzyme liquid. Purified ACE derived from rabbit lung (Sigma Co., Ltd., 0.25 unit), alternatively, was used.

0.030 mL of the various concentrations of the dipeptide composition solutions were taken into test tubes, respectively, and then, 0.1 mL of the angiotensin converting enzyme liquid as above was added into the test tubes to react with the composition at 37° C. for 5 minutes. Subsequently, 0.25 mL of hippuryl histidyl leucine (PEPTIDE Lab., Bz-Gly-His-Leu.H₂O, the final concentration of 5 mM, including 300 mM of NaCl) as a substrate was added to the test tubes to react with the enzyme at 37° C. for 30 minutes. 0.25 mL of 1N hydrochloric acid, then, was added to the test tube to terminate the reaction, 1.5 mL of ethyl acetate was added to the test tube and stirred with the vortex mixer for 20 seconds, followed by centrifuging (3000 rotations, for 5 minutes) and 1 mL of ethyl acetate layer was isolated. The layer was heated at 105° C. and for 30 minutes (aluminum block) before it was resolved in 3 mL of distilled water and the absorbance value at 228 nm of hippuric acid extracted in ethyl acetate, as the enzyme activity, was measured.

The inhibitory rate was calculated according to the following formula. A: the absorbance value at 228 nm in the case of the absence of the inhibitory agent. B: the absorbance value at 228 nm in the case of the presence of the inhibitory agent. The concentration of the dipeptide defined in the invention of this application when the inhibitory rate was 50%, was IC₅₀ value.

The inhibitory=[1−(A−a)/(B−b)]×100

A: the addition of the test sample

a: the addition of the test sample, the addition of the buffer instead of the enzyme

B: the addition of the distilled water instead of the test sample

b: the addition of the distilled water instead of the test sample, the addition of the buffer instead of the enzyme

### Example 1

### (a) The Industrial Production of the Peptide Composition Having the ACE Inhibitory Activity (1)

10000 L of water was added to 1000 kg of KATSUOBUSHI protein and these were heated (at 95° C., for 35 minutes), and then, amino acid and the water-soluble protein were removed, 2884 L of water was added to the resulting 1153 kg of the hot water-extraction residue (the protein 576 kg), pH was adjusted to 7 with 6N sodium hydroxide, and subsequently, and 2.0 wt. % (the enzyme amount: per the protein) of PROTIN NY100 (AMANO ENZYME Co., Ltd.) enzyme was added to the solution to react with it for 20 hours at 50° C. with the agitation. PH was adjusted to 6.8 by adding sodium hydroxide to the solution after the reaction and the enzyme was inactivated for 15 minutes at 98° C. The undecomposed protein was removed with the vibrating screen, the deraval and the centrifugal machine, the resulting supernatant was filtrated and clarified with the celite to obtain the decomposition liquid (the protein 200 kg). The ACE inhibitory activity, IC₅₀ value, was 0.136 mg/mL (protein).

200 kg of the peptide was loaded to the hydrophobic chromatography.

The condition for practice of the hydrophobic chromatography was explained as below.
The column loading amount: 50 kg
The column: SP-207 (column of 1000 L volume: NIPPON RENSUI Co., Ltd.)
The eluent: ethanol solution of 0.50% concentration
The flow rate: 2000 L/hour
The cycle: 4 cycles

The elution from the column filled with the hydrophobic adsorptive resin was separated to two fractions depending on the alcohol concentration, and two fractions each of 8000 L, respectively, were separated. As a result of ACE inhibitory activity measurement of each fractions according to the method described in the Test Example 1, the ACE inhibitory activity values, IC₅₀, for the elution fraction with 0% ethanol and the fraction with 50% ethanol, were “not detected” and “0.047 mg/mL”, respectively. The fractions were repeatedly isolated for four cycles, and 74.9 kg of the protein were obtained.

The ACE inhibitory activity fraction obtained with the hydrophobic chromatography (the elution fraction with 50% ethanol) was passed through the ultrafiltration membrane (produced by GE co., the module 7.9 inch×40 inch×two columns, the filtration area 48.4 m², the molecular weight 1000, the pressure 1.8 MPa, the model number GE8040F1002) to obtain twenty-fold concentrated liquid as the impermeable liquid. As a result of the measurement for the ACE inhibitory activity of the impermeable and permeable liquids, the values were 0.034 mg/mL for the permeable liquid and 0.110 mg/mL for the impermeable liquid.

The permeable liquid was concentrated under the reduced pressure and spray-dried to bulk-produce 47.6 kg of the protein powder. The yield and the like were shown in Table 1.

TABLE 1
The yield and the ACE inhibitory specific activity and all activity
of the protein for each purification process in the industrial production

| | | ACE inhibitory | |
|---|---|---|---|
| | Yield | activity (IC₅₀) | Recovery rate |
| Process | (kg-protein) | (μg-protein/mL) | (%) |
| Decomposed liquid | 200 | 136.25 | 100 |
| Resin-bound liquid | 74 | 47.42 | 107 |
| Resin-unbound liquid | 125 | Not detected | 0 |
| Impermeable liquid | 27 | 110.28 | 16.9 |
| Permeable liquid | 47 | 33.64 | 96.4 |

On the other hand, the artificial digestion was conducted for the permeable liquid and the impermeable liquid with pepsin, trypsin and chymotrypsin in order to certain the degree of the decomposition due to the digestive enzyme in vivo when orally ingesting the liquids (4 hours decomposition with pepsin, trypsin and chymotrypsin). The results were shown in Table 2.

TABLE 2
The change of the ACE inhibitory activity value after the artificial
digestion test for the permeable liquid and the impermeable liquid

| | | ACE inhibitory | Recovery |
|---|---|---|---|
| | | activity (IC₅₀) | rate |
| Fraction | The artificial digestion | (mg-protein/mL) | (%) |
| Impermeable | Not treated | 110.28 | 100 |
| liquid | Decomposed with pepsin, | 68.38 | 161.30 |
| | trypsin and chymotrypsin | | |
| Permeable | Not treated | 33.64 | 100 |
| liquid | Decomposed with pepsin, | 31.83 | 105.69 |
| | trypsin and chymotrypsin | | |

It was deduced that the permeable liquid had the potential for the digestive enzyme resistance because the liquid showed the high ACE inhibitory activity. Even though the impermeable liquid showed higher ACE inhibitory activity (IC₅₀) by 1.6 times than it before the digestion, the activity does not reach to the one for the permeable liquid.

The molecular weight was analyzed by HPLC method under the below condition (Table 3), and the result was shown in Table 4.

TABLE 3
The analysis condition of HPLC method

| | Superdex peptide |
|---|---|
| Column | (diameter 10 mm/length 300 mm) |
| Moving phase | 30% acetonitrile (0.1% TFA) |
| Flow rate | 0.5 mL/min. |
| Detection | UV (214 nm) |

TABLE 4
Molecular fractionation of the each fraction by HPLC method

| | | | Resin |
|---|---|---|---|
| | Enzymatic | | desorption |
| | decomposition | Resin desorption | permeable |
| | liquid | impermeable liquid | liquid |
| Max molecular | 5000 | 5000 | 1500 |
| weight | | | |
| Area of the | 66.78 | 40.12 | 79.75 |
| molecular weight of | | | |
| 1000 or less (%) | | | |

In the result, the maximum molecular weight of both the decomposition liquid and the resin desorption impermeable liquid were 5000.

The maximum molecular weight of the objective resin desorption permeable liquid was 1500, the rate of M.W.1000 or less among them was 79.75%.

The recovery rate in the purification of the dipeptide was shown in below Tables 5 to 7.

TABLE 5
The purification yield of tryptophan-leucine in the resin adsorption
and the ultrafiltration membrane

| | | | Protein yield |
|---|---|---|---|
| | Yield (%) | Not recovered (%) | (%) |
| Resin treatment | 100 | 0 | 40 |
| Ultrafiltration membrane | 90 | 10 | 63 |
| treatment | | | |

The recovery rate of tryptophan-leucine in the yield of 40% of the protein was 100% owing to the resin treatment for the enzyme filtrate.

The loss rate of tryptophan-leucine was 10% in the yield of 63% of the protein and the liquid amount of the concentrate side 1/20, owing to further the ultrafiltration treatment. Therefore, the yield of the purified protein was 25.2%, and the recovery rate of tryptophan-leucine was 90%.

TABLE 6
The purification yield of valine- tryptophan in the resin adsorption
and the ultrafiltration membrane

| | Yield | Not recovered | Protein yield |
|---|---|---|---|
| | (%) | (%) | (%) |
| Resin treatment | 100 | 0 | 39.98 |
| Ultrafiltration membrane treatment | 90 | 10 | 67.66 |

The recovery rate of valine-tryptophan in the yield of 39.98% of the protein was 100% owing to the resin treatment for the enzyme filtrate.

The loss rate of valine-tryptophan was 10% in the yield of 67.66% of the protein and the liquid amount of the concentrate side 1/20, owing to further the ultrafiltration treatment. Therefore, the yield of the purified protein was 27.05%, and the recovery rate of valine-tryptophan was 90%.

TABLE 7
The purification yield of alanine-tryptophan in the resin adsorption
and the ultrafiltration membrane

| | Yield | Not recovered | Protein yield |
|---|---|---|---|
| | (%) | (%) | (%) |
| Resin treatment | 100 | 0 | 39.98 |
| Ultrafiltration membrane treatment | 90 | 10 | 67.66 |

The recovery rate of alanine-tryptophan in the yield of 39.98% of the protein was 100% owing to the resin treatment for the enzyme filtrate.

The loss rate of alanine-tryptophan was 10% in the yield of 67.66% of the protein and the liquid amount of the concentrate side 1/20, owing to further the ultrafiltration treatment. Therefore, the yield of the purified protein was 27.05%, and the recovery rate of alanine-tryptophan was 90%.

The production scale for the bulk production of KATSUOBUSHI peptide was shown in Table 8.

TABLE 8
The scale for the industrial production of KATSUOBUSHI peptide

| Production quantity (kg) | Scale 1 | Scale 2 | Scale 3 |
|---|---|---|---|
| Daily production | 60 | 139 | 278 |
| Monthly production | 180 | 417 | 833 |
| Annual production | 2160 | 5000 | 10000 |

In the result, it was clear that the industrial production was possible in any of Scales 1 to 3.

(b-1) The Isolation of 5 Dipeptides-1

KATSUOBUSHI was enzymatically decomposed with PROTIN NY100 enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the amino acid analysis and TOF MS analysis for each the fractions, the peptides of each fractions were turned out to be tryptophan-leucine, leucine-tryptophan and tryptophan-isoleucine dipeptides.

KATSUOBUSHI was enzymatically decomposed with PROTIN NY100 enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 5% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the re-chromatography by use of 1.25% CH₃CN in 0.1% TFA as the moving phase, the amino acid analysis and TOF MS analysis for each the fractions, the peptides of each fractions were turned out to be valine-tyrosine and tryptophan-asparagine dipeptides.

The residue after the extract of KATSUOBUSHI with hot water was decomposed with PROTIN NY100 (AMANO ENZYME Co., Ltd.) or THERMOASE PC10F (DAIWA KASEI Co., Ltd.) enzyme, 5 peptides were isolated from said resulting decomposition substance, and ACE inhibitory value of them were shown in Table 9.

TABLE 9
The ACE inhibitory activity value of the isolated peptides

| | ACE inhibitory activity (IC₅₀: μM) |
|---|---|
| Tryptophan-isoleucine | 33.05 |
| Leucine-tryptophan | 22.97 |
| Tryptophan-leucine | 25.14 |
| Valine-tyrosine | 11.02 |
| tryptophan-asparagine | 580.2 |

(b-2) The Isolation of 5 Dipeptides-2

KATSUOBUSHI was enzymatically decomposed with PROTIN NY100 enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
   Moving phase: 15% CH₃CN in 0.1% TFA
   Flow rate: 0.2 mL/min
   Temperature: 40° C.
   Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the amino acid analysis and TOF MS analysis for each the fractions, the peptides of each fractions were turned out to be valine-tryptophan, tryptophan-tyrosine, tryptophan-methionine, methionine-tryptophan and isoleucine-tryptophan.

The peak was isolated with the change of the concentration of the moving phase in order to further certain the isolation for the fractions obtained as above.
Column: Acquity UPLC BEH C18 (2.1 mmID×100 mmL, 1.7 μm)
Moving phase: 10% CH₃CN in 0.1% TFA
Flow rate: 0.2 mL/min
Temperature: 40° C.
Detection: UV 200-300 nm

As a result of the HPLC analysis, the fractions were turned out to be the degree of the purity of 95% or more.

The residue after the extract of KATSUOBUSHI with hot water was decomposed with PROTIN NY100 (AMANO ENZYME Co., Ltd.) enzyme, 5 peptides were isolated from said resulting decomposition substance, and ACE inhibitory value of them were shown in Table 10.

TABLE 10
The ACE inhibitory activity value of the isolated peptides

| | ACE inhibitory activity (IC₅₀: μM) |
|---|---|
| Valine- tryptophan | 1.60 |
| Tryptophan- tyrosine | 57.00 |
| Tryptophan-methionine | 36.48 |
| Methionine- tryptophan | 3.80 |
| Isoleucine-tryptophan | 2.00 |

(b-3) The Isolation of 5 Dipeptides-3

KATSUOBUSHI was enzymatically decomposed with PROTIN NY100 enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the amino acid analysis and TOF MS analysis for each the fractions, the peptides of each fractions were turned out to be a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

The residue after the extract of KATSUOBUSHI with hot water was decomposed with PROTIN NY100 (AMANO ENZYME Co., Ltd.) enzyme, 5 peptides were isolated from said resulting decomposition substance, and ACE inhibitory value of them were shown in Table 11.

TABLE 11
The ACE inhibitory activity value of the isolated peptides

| | ACE inhibitory activity (IC₅₀: μM) |
|---|---|
| Serine-tryptophan | 22.10 |
| Asparagine-tryptophan | 25.00 |
| Glutamine-tryptophan | 32.60 |
| Glycine- tryptophan | 30.00 |
| Alanine-tryptophan | 10.00 |

As described above, it was confirmed that the composition obtained in Example 1 comprised the dipeptides shown in Tables 9 to 11.

### Example 2

### (a) The Industrial Production of the Peptide Composition Having the ACE Inhibitory Activity

10000 L of water was added to 1000 kg of KATSUOBUSHI protein and these were heated (at 95° C., for 35 minutes), and then, amino acid and the water-soluble protein were removed, 4436 L of water was added to the resulting 1774 kg of the hot water-extraction residue (the protein 886 kg), pH was adjusted to 7 with 6N sodium hydroxide, and subsequently, and 1.0 wt. % (the enzyme amount: per the protein) of THERMOASE PC10F (DAIWA KASEI Co., Ltd.) enzyme was added to the solution to react with it for 17 hours at 50° C. with the agitation. PH was adjusted to 6.8 by adding sodium hydroxide to the solution after the reaction and the enzyme was inactivated for 15 minutes at 98° C. The undecomposed protein was removed with the vibrating screen, the deraval and the centrifugal machine, the resulting supernatant was filtrated with the celite to obtain the enzymatically decomposition liquid (the protein 200 kg). The ACE inhibitory activity, IC₅₀ value, was 0.204 mg/mL (protein).

200 kg of the peptide was loaded to the hydrophobic chromatography.

The condition for practice of the hydrophobic chromatography was explained as below.
The column loading amount: 50 kg
The column: SP-207 (column of 1000 L volume: NIPPON RENSUI Co., Ltd.)
The eluant: ethanol solution of 0.50% concentration
The flow rate: 2000 L/hour
The cycle: 4 cycles

The elution from the column filled with the hydrophobic adsorptive resin was separated to two fractions depending on the alcohol concentration, and two fractions each of 8000 L, respectively, were isolated. As a result of the measurement according to the method described in the Test Example 1 for the ACE inhibitory activity of each fractions, the ACE inhibitory activity values, IC₅₀, for the elution fraction with 0% ethanol and the fraction with 50% ethanol, were “not detected” and “0.071 mg/mL”, respectively. The fractions were repeatedly isolated for four cycles, and 74.0 kg of the protein were obtained.

The ACE inhibitory activity fraction obtained with the hydrophobic chromatography (the elution fraction with 50% ethanol) was passed through the ultrafiltration membrane (produced by GE co., the module 7.9 inch×40 inch×two columns, the filtration area 48.4 m², the molecular weight 1000, the pressure 1.8 MPa, the model number GE8040F1002) to obtain twenty-fold concentrated liquid as the impermeable liquid. As a result of the measurement for the ACE inhibitory activity of the impermeable and permeable liquids, the values were 0.050 mg/mL for the permeable liquid and 0.165 mg/mL for the impermeable liquid, respectively.

The permeable liquid was concentrated under the reduced pressure and spray-dried to obtain bulk-produce 71.4 kg of the protein powder. The yield and the like were shown in Table 12.

TABLE 12
The yield and the ACE inhibitory specific activity and all activity
of the protein for each purification process in the industrial production

| | | ACE inhibitory | Recovery |
|---|---|---|---|
| | Yield | activity (IC₅₀) | rate |
| Process | (kg-protein) | (μg-protein/mL) | (%) |
| Decomposed liquid | 200 | 204.00 | 100 |
| Resin-bound liquid | 74 | 47.4271.13 | 106 |
| Resin-unbound liquid | 126 | Not detected | 0 |
| Impermeable liquid | 25.9 | 165.42 | 16.0 |
| Permeable liquid | 48.1 | 50.46 | 97.3 |

On the other hand, the artificial digestion was conducted for the permeable liquid and the impermeable liquid with pepsin, trypsin and chymotrypsin in order to certain the degree of the decomposition due to the digestive enzyme in vivo when orally ingesting the liquids (4 hours decomposition with pepsin, trypsin and chymotrypsin). The result was shown in Table 13.

TABLE 13
The change of the ACE inhibitory activity value after the artificial
digestion test for the permeable liquid and the impermeable liquid

| | | ACE inhibitory | Recovery |
|---|---|---|---|
| | | activity (IC₅₀) | rate |
| Fraction | The artificial digestion | (μg-protein/mL) | (%) |
| Impermeable | Not treated | 165.42 | 100 |
| liquid | Decomposed with pepsin, | 110.28 | 150 |
| | trypsin and chymotrypsin | | |
| Permeable | Not treated | 50.46 | 100 |
| liquid | Decomposed with pepsin, | 50.00 | 101 |
| | trypsin and chymotrypsin | | |

It was deduced that the permeable liquid had the potential for the digestive enzyme resistance because the liquid showed the high ACE inhibitory activity. Even though the impermeable liquid showed higher ACE inhibitory activity (IC₅₀) by 1.5 times than it before the digestion, the activity does not reach to the one for the permeable liquid.

The molecular weight was analyzed by HPLC method under the below condition (Table 14), and the result was shown in Table 15.

TABLE 14
The analysis condition of HPLC method

| | Superdex peptide |
|---|---|
| Column | (diameter 10 mm/length 300 mm) |
| Moving phase | 30% acetonitrile (0.1% TFA) |
| Flow rate | 0.5 mL/min. |
| Detection | UV (214 nm) |

TABLE 15
Molecular fractionation of the each fraction by HPLC method

| | Enzymatic | | |
|---|---|---|---|
| | decomposition | Resin desorption | Resin desorption |
| | liquid | impermeable liquid | permeable liquid |
| Max molecular | 5000 | 5000 | 1500 |
| weight | | | |
| Area of the | 66.8 | 40.1 | 79.8 |
| molecular | | | |
| weight of | | | |
| 1000 or less | | | |
| (%) | | | |

In the result, the maximum molecular weight of both the decomposition liquid and the resin desorption impermeable liquid were 5000.

The maximum molecular weight of the objective resin desorption permeable liquid was 1500, the rate of M.W.1000 or less among them was 79.8%.

The production scale for the bulk production of KATSUOBUSHI peptide was shown in Table 16.

TABLE 16
The scale for the industrial production of KATSUOBUSHI peptide

| Production quantity (kg) | Scale 1 | Scale 2 | Scale 3 |
|---|---|---|---|
| Daily production | 61 | 139 | 278 |
| Monthly production | 183 | 417 | 833 |
| Annual production | 2196 | 5000 | 10000 |

In the result, it was clear that the industrial production was possible in any of Scales 1 to 3.

(b-1) The Isolation of Five Dipeptides-1

KATSUOBUSHI was enzymatically decomposed with THERMOASE PC10F (DAIWA KASEI Co., Ltd.) enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the amino acid analysis and TOF MS analysis for each the fractions, the peptides of each fractions were turned out to be tryptophan-leucine, leucine-tryptophan and tryptophan-isoleucine dipeptides.

KATSUOBUSHI was enzymatically decomposed with THERMOASE PC10F (DAIWA KASEI Co., Ltd.), resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 5% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the re-chromatography by use of 1.25% CH₃CN in 0.1% TFA as the moving phase, the amino acid analysis and TOF MS analysis for each the fractions, the peptides of each fractions were turned out to be valine-tyrosine and tryptophan-asparagine dipeptides.

(b-2) The Isolation of Five Dipeptides-2

KATSUOBUSHI was enzymatically decomposed with THERMOASE PC10F (DAIWA KASEI Co., Ltd.) enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the amino acid analysis and TOF MS analysis for each the fractions, the peptides of each fractions were turned out to be a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence and a dipeptide composed of a isoleucine-tryptophan sequence.

(b-3) The Isolation of Five Dipeptides-3

KATSUOBUSHI was enzymatically decomposed with THERMOASE PC10F (DAIWA KASEI Co., Ltd.) enzyme, resulting liquid was subject to the column adsorption and subsequently the alcohol desorption, obtained fraction was passed through the ultrafiltration membrane, to obtain the permeable liquid (KATSUOBUSHI peptide), and then, the dipeptides in the liquid was isolated.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

Each one fraction was isolated per 30 seconds under the condition described above. Each fraction were evaporated to dryness under the reduced pressure, and then, the ACE inhibitory activity thereof was measured according to the method described above. In the result, high ACE inhibitory activity was recognized in the peptide fraction. After the fractions were lyophilized, respectively, trace of peptide was obtained. As a result of the amino acid analysis and TOF MS analysis for each the fractions, the peptides of each fractions were turned out to be a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

### Example 3

### The Synthesis of Peptide by Use of the Synthesis Method

The peptide chain was sequentially extended from C-terminal by use of BOC method with the peptide auto synthesis machine made by APPLIED BIO SYSTEMS Co., Ltd. (ABI 430 model) according to the program to synthesize the objective protected peptide resin.

After the peptide construction onto the resin was completed, the protected peptide resin was dried. The deprotection of the resulting protected peptide and the separation of the peptide from the resin carrier, are treated with anhydrous hydrogen fluoride treatment (HF/p-Creso18:2 v/v, 60 minutes). The resulting crude peptide was extracted with 90% acetic acid, and lyophilized to the powder solid. The obtained crude peptide was purified by loading it to the high-performance liquid chromatography equipped with ODS column to obtain the objective peptide.
Column: YMC-Pack ODS-A (30 mm ID×250 mmL, YMC)
Moving phase: Buffer A: 5% CH₃CN, 0.1% TFA
   Buffer B: 40% CH₃CN, 0.1% TFA
Gradient: 0 to 10 min: 0% Buffer B
   10 to 90 min: 0 to 100% Buffer B
Flow rate: 20 mL/min
Detection: UV 220 nm

The purity of the purified peptide was evaluated with the high-performance liquid chromatography equipped with ODS column.
Column: Zorbax 300SB-C18 (4.6 mm ID×150 mmL, Agilent Technologies)
Moving phase: Buffer A: 1% CH₃CN, 0.1% TFA
   Buffer B: 60% CH₃CN, 0.1% TFA
Gradient: 0 to 25 min: 0 to 100% Buffer B
Flow rate: 1 mL/min
Detection: UV 220 nm
   (i-1) The Synthesis of Tryptophan-Leucine Dipeptide:

The peptide chain was expanded by using Boc-Leu (BrZ) resin (0.5 mmol) as a starting amino acid resin carrier and 2 mM of amino acid derivative Boc-Trp. The purification was performed by means of the method described above to obtain purified tryptophan-leucine. The result of the measurement for the purity of the purified substance as described above was 94.06%.

(i-2) The Synthesis of Leucine-Tryptophan Dipeptide:

The peptide chain was expanded by using Boc-Trp (BrZ) resin (0.5 mmol) as a starting amino acid resin carrier and 2 mM of amino acid derivative Boc-Leu. The purification was performed by means of the method described above to obtain purified leucine-tryptophan. The result of the measurement for the purity of the purified substance as described above was 88.84%.

(i-3) The Synthesis of Tryptophan-Isoleucine Dipeptide:

The peptide chain was expanded by using Boc-ILe (BrZ) resin (0.5 mmol) as a starting amino acid resin carrier and 2 mM of amino acid derivative Boc-Trp. The purification was performed by means of the method described above to obtain purified tryptophan-isoleucine. The result of the measurement for the purity of the purified substance as described above was 95.00%.

(i-4) The Synthesis of Valine-Tyrosine Dipeptide:

The peptide chain was expanded by using Boc-Tyr (BrZ) resin (0.5 mmol) as a starting amino acid resin carrier and 2 mM of amino acid derivative Boc-Val. The purification was performed by means of the method described above to obtain purified valine-tyrosine. The result of the measurement for the purity of the purified substance as described above was 95.00%.

(i-5) The Synthesis of Tryptophan-Asparagine Dipeptide:

The peptide chain was expanded by using Boc-Asn (BrZ) resin (0.5 mmol) as a starting amino acid resin carrier and 2 mM of amino acid derivative Boc-Trp. The purification was performed by means of the method described above to obtain purified tryptophan-asparagine dipeptide. The result of the measurement for the purity of the purified substance as described above was 95.00%.

(ii-1) The Synthesis of Valine-Tryptophan Dipeptide:

The peptide chain was expanded by using Boc-tryptophan (BrZ) resin (0.5 mmol) as a starting amino acid resin carrier and 2 mM of amino acid derivative Boc-valine. The purification was performed by means of the method described above to obtain purified valine-tryptophan.

(ii-2) The Synthesis of Tryptophan-Tyrosine Dipeptide:

The peptide chain was expanded by using Boc-tyrosine (BrZ) resin (0.5 mmol) as a starting amino acid resin carrier and 2 mM of amino acid derivative Boc-tryptophan. The purification was performed by means of the method described above to obtain purified tryptophan-tyrosine.

(ii-3) The Synthesis of Tryptophan-Methionine Dipeptide:

The peptide chain was expanded by using Boc-tryptophan (BrZ) resin (0.5 mmol) as a starting amino acid resin carrier and 2 mM of amino acid derivative Boc-methionine. The purification was performed by means of the method described above to obtain purified methionine-tryptophan. The result of the measurement for the purity of the purified substance described above was 95.00%.

(ii-4) The Synthesis of Methionine-Tryptophan Dipeptide:

The peptide chain was expanded by using Boc-tryptophan (BrZ) resin (0.5 mmol) as a starting amino acid resin carrier and 2 mM of amino acid derivative Boc-methionine. The purification was performed by means of the method described above to obtain purified tryptophan-methionine.

(ii-5) The Synthesis of Isoleucine-Tryptophan Dipeptide:

The peptide chain was expanded by using Boc-tryptophan (BrZ) resin (0.5 mmol) as a starting amino acid resin carrier and 2 mM of amino acid derivative Boc-isoleucine. The purification was performed by means of the method described above to obtain purified isoleucine-tryptophan.

(iii-1) The Synthesis of Serine-Tryptophan Dipeptide:

The peptide chain was expanded by using Boc-tryptophan (BrZ) resin (0.5 mmol) as a starting amino acid resin carrier and 2 mM of amino acid derivative Boc-serine. The purification was performed by means of the method described above to obtain purified serine-tryptophan.

(iii-2) The Synthesis of Asparagine-Tryptophan Dipeptide:

The peptide chain was expanded by using Boc-tryptophan (BrZ) resin (0.5 mmol) as a starting amino acid resin carrier and 2 mM of amino acid derivative Boc-asparagine. The purification was performed by means of the method described above to obtain purified asparagine-tryptophan.

(iii-3) The Synthesis of Glutamine-Tryptophan Dipeptide:

The peptide chain was expanded by using Boc-tryptophan (BrZ) resin (0.5 mmol) as a starting amino acid resin carrier and 2 mM of amino acid derivative Boc-glutamine. The purification was performed by means of the method described above to obtain purified glutamine-tryptophan.

(iii-4) The Synthesis of Glycine-Tryptophan Dipeptide:

The peptide chain was expanded by using Boc-tryptophan (BrZ) resin (0.5 mmol) as a starting amino acid resin carrier and 2 mM of amino acid derivative Boc-glycine. The purification was performed by means of the method described above to obtain purified glycine-tryptophan.

(iii-5) The Synthesis of Alanine-Tryptophan Dipeptide:

The peptide chain was expanded by using Boc-tryptophan (BrZ) resin (0.5 mmol) as a starting amino acid resin carrier and 2 mM of amino acid derivative Boc-alanine. The purification was performed by means of the method described above to obtain purified alanine-tryptophan.

### Example 4-1

The broth beverage which has composition as described below was produced by use of the dipeptide isolate as obtained in Example 1 (b-1).

### (a) Material and Compounded Amount:

500 mL of the extraction liquid of KATSUOBUSHI with hot water (the noodle broth) and the mixture of five dipeptides isolated in Example 1 (b-1) (43 mg of tryptophan-leucine dipeptide, 30 mg of leucine-tryptophan dipeptide, 16 mg of tryptophan-isoleucine dipeptide, 36 mg of valine-tyrosine dipeptide and 40 mg of tryptophan-asparagine dipeptide).

### (b) Production Method:

KATSUOBUSHI was processed with hot water extraction (95° C., 35 minutes), resulting liquid was filtrated with celite and the filtrate was cooled to the room temperature. The mixture of five dipeptides as described above was added to the cooled extracting liquid, and the liquid agitated to dissolve the peptides. Thereby, the broth beverage was produced.

### Example 4-2

The broth beverage which has composition as described below was produced by use of the dipeptide isolate as obtained in Example 1 (b-2).

### (a) Material and Compounded Amount:

500 mL of the extraction liquid of KATSUOBUSHI with hot water (the noodle broth) and the mixture of five dipeptides isolated in Example 1 (b-2) (58.89 mg of valine-tryptophan dipeptide, 13.40 mg of tryptophan-tyrosine dipeptide, 20.00 mg of tryptophan-methionine dipeptide, 13.16 mg of methionine-tryptophan dipeptide and 39.20 mg of isoleucine-tryptophan dipeptide).

### (b) Production Method:

KATSUOBUSHI was processed with hot water extraction (95° C., 35 minutes), resulting liquid was filtrated with celite and the liquid was cooled to the room temperature. The mixture of five dipeptides as described above was added to the cooled extracting liquid, and the liquid agitated to dissolve the peptides. Thereby, the broth beverage was produced.

### Example 4-3

The broth beverage which has composition as described below was produced by use of the dipeptide isolate as obtained in Example 1 (b-3).

### (a) Material and Compounded Amount:

500 mL of the extraction liquid of KATSUOBUSHI with hot water (the noodle broth) and the mixture of five dipeptides isolated in Example 1 (b-3) (19.10 mg of serine-tryptophan dipeptide, 23.30 mg of asparagine-tryptophan dipeptide, 31.72 mg of glutamine-tryptophan dipeptide, 86.30 mg of glycine-tryptophan dipeptide and 55.68 mg of alanine-tryptophan dipeptide).

### (b) Production Method:

KATSUOBUSHI was processed with hot water extraction (95° C., 35 minutes), resulting liquid was filtrated with celite and the liquid was cooled to the room temperature. The mixture of five dipeptides as described above was added to the cooled extracting liquid, and the liquid agitated to dissolve the peptides. Thereby, the broth beverage was produced.

### Example 5

(a) The quantitative determination for dipeptide in the reacting mixture which was obtained by decomposing the hot water extraction residue of KATSUOBUSHI protein by use of PROTIN NY100 enzyme.

2000 mL of water was added to 160 g of KATSUOBUSHI protein and the extraction with hot water (95° C., 35 minutes) was processed. To obtained residue (the insoluble protein), 10 times volume of water was added, pH was adjusted to 7, PROTIN NY100 (AMANO ENZYME CO., Ltd.) enzyme was added to the liquid owing to the reaction at 50° C. for 20 hours, pH was adjusted to 6.8, the heating was done, and then, the vibrating screen, the decanter, the deraval, the sharpless treatment, and the celite filtration were done, and further, the vacuum concentration and the spray drying were done.

The resulting powder was loaded to the hydrophobic chromatography, the elution was done with 250 mL of water, and subsequent 250 mL of 50% ethanol, the high activity fraction was obtained in the 50% ethanol eluent. The subsequent permeable fraction, obtained by the ultrafiltration (the membrane having molecular weight 1000) of the high active fraction, was concentrated under the reduced pressure (to solid content 40%), and then, was subjected to the spray drying (the inlet temperature: 150 to 200° C., the outlet temperature: 50 to 90° C.) to obtain 500 mg of the high activity powder.

The functional food was obtained by blending 500 mg of the powder as described above (KATSUOBUSHI peptide) as the material. The powder could be formulated into beverage, tablet, soup and the like, as the processed food. The formulation ratio of the food into which KATSUOBUSHI peptide was formulated was shown in Table 17.

TABLE 17
The formulation example of the processed food (the tablet sweets)
into which KATSUOBUSHI peptide was formulated

| Material | Formulation amount (kg) |
|---|---|
| KATSUOBUSHI peptide | 50 |
| Sweetener | 10 |
| Stevia | 5 |
| Acidulant | 100 |
| Maltodextrin | 500 |
| Fragrance | 50 |

Regarding the production to tablet sweets, tablet and capsule of KATSUOBUSHI peptide, KATSUOBUSHI peptide and food materials were measured, admixed, granulated, tableted and coated, and then, the ACE inhibitory activity and the dipeptide content thereof were measured, and the standard product was produced.

### Example 6-1

The quantitative determination was conducted as follows, for tryptophan-leucine, leucine-tryptophan, tryptophan-isoleucine, valine-tyrosine and tryptophan-asparagine. That is to say, the quantitative determination of these dipeptides was conducted as follows, from the powder or the processed product.

### Sep-Pak C18 Pretreatment:

The enzymatic decomposition product which was produced by enzymatically decomposing a hot water extraction residue of KATSUOBUSHI and the processed food thereof were weighed for 25 mg and 5 g, respectively, were loaded to Sep-Pak C18 cartridge, the water soluble fraction was removed from them, and the bound fraction was eluted with 50% ethanol was obtained as the liquid sample.

8000 μg of the purified ACE inhibitory peptide which was recycled from the sample obtained with Sep-Pak C18 treatment as described above, was dissolved in 100 μL of the purified water, and 2000 μg/25 μL thereof was loaded to the high-performance liquid chromatography equipped with C-18 column to fractionate the peptide. The conditions were shown as follows.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

1 μg/μL of the synthesized tryptophan-leucine, leucine-tryptophan and tryptophan-isoleucine were loaded as an authentic preparation, respectively. The elution time of tryptophan-leucine, leucine-tryptophan and tryptophan-isoleucine were 28.61, 20.65 and 20.32 minutes, respectively.

As the result of the quantitative determination, the contents of tryptophan-leucine, leucine-tryptophan and tryptophan-isoleucine were 43 mg, 30 mg and 16 mg in the KATSUOBUSHI peptide, respectively.

1 μg/μL of the synthesized valine-tyrosine and tryptophan-asparagine were loaded as an authentic preparation, respectively, under the column conditions as described below.
Column: Acquity UPLC BEH C18 (2.1 mmID×100 mmL, 1.7 μm)
Moving phase: 5% CH₃CN in 0.1% TFA
Flow rate: 0.2 mL/min
Temperature: 40° C.
Detection: UV 200-300 nm

The elution time of valine-tyrosine and tryptophan-asparagine were 9.56 minutes. Further, re-chromatography was done under the conditions as described below.
Column: Acquity UPLC BEH C18 (2.1 mmID×100 mmL, 1.7 μm)
Moving phase: 1.25% CH₃CN in 0.1% TFA
Flow rate: 0.2 mL/min
Temperature: 40° C.
Detection: UV 200-300 nm

The elution time of valine-tyrosine and tryptophan-asparagine were 35.60 minutes and 28.92 minutes, respectively.

The quantitative value was calculated on the basis of the peak areas of dipeptides isolated from KATSUOBUSHI peptide and the authentic preparation.

As the result of the quantitative determination, the contents of valine-tyrosine and tryptophan-asparagine were 36 mg and 40 mg, respectively, in KATSUOBUSHI peptide.

### Example 6-2

The quantitative determination was conducted as follows, for valine-tryptophan, tryptophan-tyrosine, tryptophan-methionine, methionine-tryptophan and isoleucine-tryptophan. That is to say, the quantitative determination of these dipeptides was conducted as follows, from the powder or the processed product.

### Sep-Pak C18 Pretreatment:

The enzymatic decomposition product which was produced by enzymatically decomposing a hot water extraction residue of KATSUOBUSHI and the processed food thereof were weighed for 25 mg and 5 g, respectively, were loaded to Sep-Pak C18 cartridge, the water soluble fraction was removed from them, and the bound fraction was eluted with 50% ethanol was obtained as the liquid sample.

8000 μg of the purified ACE inhibitory peptide which was recycled from the sample obtained with Sep-Pak C18 treatment as described above, was dissolved in 100 μL of the purified water, and 2000 μg/25 μL thereof was loaded to the high-performance liquid chromatography equipped with C-18 column to fractionate the peptide. The conditions were shown as follows.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

1 μg/μL of the synthesized valine-tryptophan, tryptophan-tyrosine, tryptophan-methionine, methionine-tryptophan and isoleucine-tryptophan were loaded as an authentic preparation, respectively. The elution time of valine-tryptophan, tryptophan-tyrosine, tryptophan-methionine, methionine-tryptophan and isoleucine-tryptophan were 10.52, 11.21, 13.84, 15.57 and 16.82 minutes, respectively.

The result of the quantitative determination was as the following Table 18.

TABLE 18
The amount of the dipeptides of the invention
in the KATSUOBUSHI peptide

| | The quantitative value (mg/100 g |
|---|---|
| | KATSUOBUSHI peptide) |
| Valine-tryptophan | 58.89 |
| Tryptophan-tyrosine | 13.40 |
| Tryptophan-methionine | 20.00 |
| Methionine-tryptophan | 13.16 |
| Isoleucine-tryptophan | 39.20 |

### Example 6-3

The quantitative determination was conducted as follows, for serine-tryptophan, asparagine-tryptophan, glutamine-tryptophan, glycine-tryptophan and alanine-tryptophan.

That is to say, the quantitative determination of these dipeptides was conducted as follows, from the powder or the processed product.

### Sep-Pak C18 Pretreatment:

The enzymatic decomposition product which was produced by enzymatically decomposing a hot water extraction residue of KATSUOBUSHI and the processed food thereof were weighed for 25 mg and 5 g, respectively, were loaded to Sep-Pak C18 cartridge, the water soluble fraction was removed from them, and the bound fraction was eluted with 50% ethanol was obtained as the liquid sample.

8000 μg of the purified ACE inhibitory peptide which was recycled from the sample obtained with Sep-Pak C18 treatment as described above, was dissolved in 100 μL of the purified water, and 2000 μg/25 μL thereof was loaded to the high-performance liquid chromatography equipped with C-18 column to fractionate the peptide. The conditions were shown as follows.
Column: Acquity UPLC BEH C18 (2.1 mmID×100 mmL, 1.7 μm)
Moving phase: 15% CH₃CN in 0.1% TFA
Flow rate: 0.2 mL/min
Temperature: 40° C.
Detection: UV 200-300 nm

1 μg/μL of the synthesized serine-tryptophan, asparagine-tryptophan, glutamine-tryptophan, glycine-tryptophan and alanine-tryptophan were loaded as an authentic preparation, respectively. The elution time of serine-tryptophan, asparagine-tryptophan, glutamine-tryptophan, glycine-tryptophan and alanine-tryptophan were 8.12, 8.28, 8.38, 8.75 and 8.92 minutes, respectively.

The result of the quantitative determination was as the following Table 19.

TABLE 19
The amount of the dipeptides of the invention
in the KATSUOBUSHI peptide

| | The quantitative value (mg/100 g |
|---|---|
| | KATSUOBUSHI peptide) |
| Serine-tryptophan | 19.10 |
| Asparagine-tryptophan | 23.30 |
| Glutamine-tryptophan | 31.72 |
| Glycine- tryptophan | 86.30 |
| Alanine-tryptophan | 55.68 |

### Example 7

### The Plant Production of ACE Inhibitory Peptide

21.9 kg of KATSUOBUSHI protein was extracted with hot water for 35 minutes at 95° C., and 5.5 kg of the resulting soluble protein was used as the broth (the noodle broth). To 16.4 kg of the water insoluble protein material, as the residue after the hot water extraction of KATSUOBUSHI, which was obtained as the by-product, water was added, the liquid was adjusted to pH7 and decomposed with PROTIN NY100 (AMANO ENZYME Co., Ltd.) enzyme for 20 hours at 50° C. The resulting reaction mixture which was enzymatically decomposed, was subjected to screen (100 mesh), the deraval (the three layers continuous discharging-centrifugal machine), the sharpless (ultracentrifuge, 15000 rotations/minute) and the celite filtration (Hyflo Super Celite 0.4%), and subsequently, the filtrate was obtained. The filtrate was spray-dried (the spray dryer, the inlet temperature 150 to 200° C., the outlet temperature 90° C. or less) to be able to obtain the powder (10 kg). Regarding the powder, IC₅₀ of the angiotensin converting enzyme inhibitory activity was 136.25 μg/m L.

The filtrate as described above (the protein 10 kg) was dissolved into 600 L of water, the liquid was loaded to the column (φ45 cm×150 cm) which was filled with the hydrophobic absorptive resin (Sepabead SP-207, MITSUBISHI CHEMICAL Co., Ltd.) and preliminarily equilibrated with water, the adsorption was conducted, and then, the elution was conducted with 600 L of water followed by 600 L of 50% ethanol solution.

Although the used hydrophobic absorptive resin was the styrene-divinylbenzene resin, any the reversed-phase partition resin or the hydrophobic absorptive resin such as octadecyl silica (YMC Co., Ltd.) could be also used. The liquid used in the elution is not limited to ethanol.

Further, 50% ethanol-eluted fraction obtained as above was passed through the ultrafiltration membrane (produced by GE Co., Ltd., the module 2.4 inch×40 inch×2 columns, the filtration area 5 m²; the membrane having the molecular weight 1000; the model number 2540F1072), and the resulting permeable liquid was concentrated under the reduced pressure (the solid content 40%) and spray-dried (the spray dryer) to obtain KATSUOBUSHI peptide.

It was considered that the angiotensin converting enzyme inhibitory peptide has the property to absorb to the hydrophobic absorptive resin, on the basis that the high activity was shown in the 50% ethanol-eluted fraction. In addition, it was deduced that the high activity peptide among the low molecular peptides existed, on the basis that the permeable liquid which permeated the ultrafiltration membrane having 1000 of molecular weight showed the high activity and the artificial digested liquid of the impermeable fraction showed the increased activity. It was possible to obtain the substance having the high-angiotensin converting enzyme inhibitory activity in good yield in the industrial production scale, by use of the purification and the column chromatography and the ultrafiltration treatment effectively in the invention, based on the abovementioned points.

### Example 8

### The Animal Test by the Intravenous Injection of KATSUOBUSHI Peptide

The male rat (SHR/Izm) was anesthetized by the intraperitoneal administration of urethane α-chloralose (1 g/kg, 50 mg/kg) mixture, and fixed on its back. The blood pressure was recorded via the pressure transducer P23×L, Spectramed Co., Ltd.) which was connected to a cannula inserted into a right femoral artery and the blood pressure amplifier (2238, NIPPON DENKI SANEI Co., Ltd.) The heart rate was measured from the blood pressure pulse wave by driving the instant counting unit (1321, NIPPON DENKI SANEI Co., Ltd.) These parameters were recorded to the pen-writing recorder (RECTI-HORIZ-8K, NIPPON DENKI SANEI Co., Ltd.) The physiological saline specified by Japanese Pharmacopeia (Otsuka Pharmaceutical Factory Inc.) was continuously injected from the left femoral artery, and the test substance was administrated from the site by use of the microsyringe. KATSUOBUSHI peptide obtained in Example 7 was used as the test substance.

TABLE 20
The construction and the single dose of each group:
3 animals per group

| | Dose | Concentration | Dose volume | Number of |
|---|---|---|---|---|
| Test group | (mg/kg) | (mg/mL) | (mL/kg) | animals |
| Low dosage | 0.1 | 1.0 | 0.1 | 3 |
| Medium dosage | 0.3 | 3.0 | 0.1 | 3 |
| High dosage | 1.0 | 10.0 | 0.1 | 3 |

As the result, the intravenous injection of 0.1, 0.3 and 1.0 mg/kg of KATSUOBUSHI peptide showed the anti-hypertensive action (Table 21, Table 22 and Table 23).

TABLE 21
The blood pressure lowering action of KATSUOBUSHI peptide
in Example 7 for the intravenous administration under the
anesthesia into SHR (0.1 mg/kg the intravenous injection)

| | | | Degree of |
|---|---|---|---|
| | Systolic blood pressure (mmHg) | | variability between |
| Animal | Before | After | before and after |
| No. | administration | administration | administration (%) |
| 07 | 140 | 104 | -26 |
| 08 | 131 | 95 | -27 |
| 01 | 136 | 99 | -27 |
| | | | Mean −27 |
| | | | SD 1 |

TABLE 22
The blood pressure lowering action of KATSUOBUSHI peptide
in Example 7 for the intravenous administration under the
anesthesia into SHR (0.3 mg/kg the intravenous injection)

| | | | Degree of |
|---|---|---|---|
| | Systolic blood pressure (mmHg) | | variability between |
| | Before | After | before and after |
| Animal No. | administration | administration | administration (%) |
| 08 | 185 | 163 | −12 |
| 01 | 124 | 104 | −16 |
| | | | Mean −14 |
| | | | SD 3 |

TABLE 23
The blood pressure lowering action of KATSUOBUSHI peptide in
Example 7 for the intravenous administration under the anesthesia
into SHR (1.0 mg/kg the intravenous injection)

| | | | Degree of |
|---|---|---|---|
| | Systolic blood pressure (mmHg) | | variability between |
| | Before | After | before and after |
| Animal No. | administration | administration | administration (%) |

| | | | |
|---|---|---|---|
| 02 | 132 | 116 | −12 |
| 08 | 154 | 122 | −21 |
| 01 | 128 | 112 | −13 |
| | | | Mean −15 |
| | | | SD 5 |

### Example 9

### The Animal Test by the Oral Administration of KATSUOBUSHI Peptide-1

Animal: SHR/Izm

Animal numbers: 32

Measurement items: Blood pressure (Systolic blood pressure) and Heart rate

Measurement time: Before, and 2, 4, 6, 8 and 24 hours after the administration

Measurement method: Noninvasively measured by use of Tail-cuffs method (the hemadynamometer for rat and mouse, MK-2000, MUROMACHI KIKAI Co., Ltd.) The blood pressure was the average of three pressure values excluding the minimum and maximum values among total five measurements, respectively. The heart rate was the average of the heart rates at the time of the measurement for blood pressure adopted.

When the abnormal value was measured due to the ramp of the rat at the time of the measurement, the measured data was not taken into the consideration, and the additional measurement was conducted.

TABLE 24
The construction and the doses of each group

| | Dose | Concentration | Dose volume | Number of |
|---|---|---|---|---|
| Test group | (mg/kg) | (mg/mL) | (mL/kg) | animals |
| Control (solvent)¹⁾ | 0 | 0 | 10 | 8 |
| Low dosage | 1 | 0.1 | 10 | 8 |
| Medium dosage | 3 | 0.3 | 10 | 8 |
| High dosage | 10 | 1 | 10 | 8 |

¹⁾Water for use in the injection was administrated.

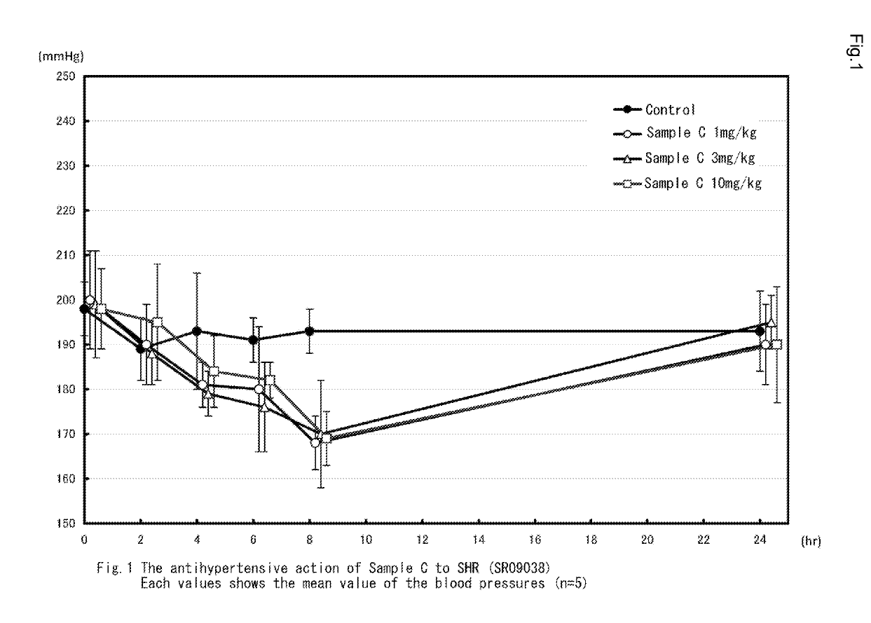

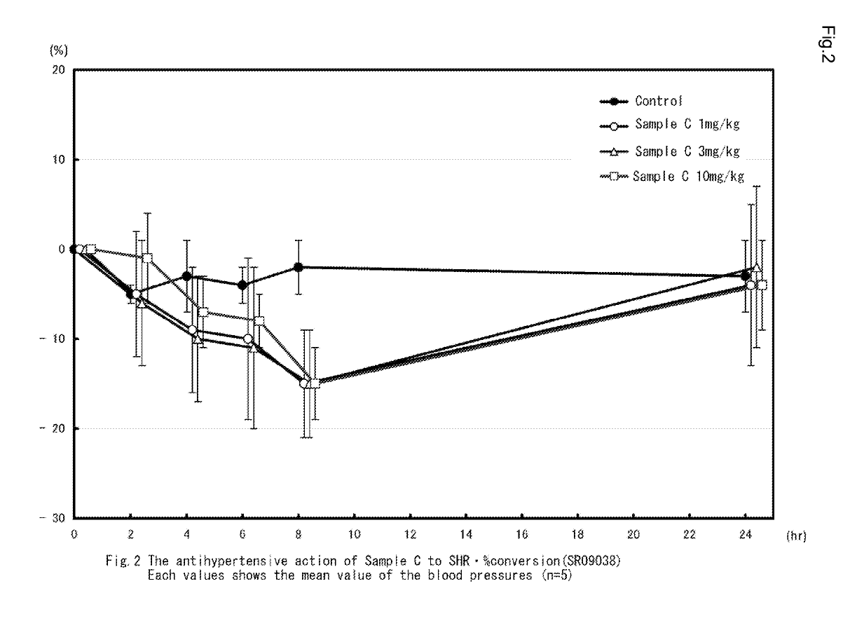

As the result, the administration of 1, 3 and 10 mg/kg of KATSUOBUSHI peptide in Example 7 (Sample C) showed the anti-hypertensive action (FIG. 1 and FIG. 2).

The animal test by the oral administration of KATSUOBUSHI peptide-2

Animal: SHR/Izm

Animal numbers: 24

Measurement items: Blood pressure (Systolic blood pressure) and Heart rate

Measurement time: Before, and 2, 4, 6, 8 and 24 hours after the administration

Measurement method: Noninvasively measured by use of Tail-cuffs method (the hemadynamometer for rat and mouse, MK-2000, MUROMACHI KIKAI Co., Ltd.) The blood pressure was the average of three pressure values. The heart rate was the average of the heart rates at the time of the measurement for blood pressure adopted.

The construction and the doses of each group was as shown in Table 25.

TABLE 25
The construction and the doses of each group

| | Dose | Concentration | Dose volume | Number of |
|---|---|---|---|---|
| Test group | (mg/kg) | (mg/mL) | (mL/kg) | animals |
| Control (solvent)¹⁾ | 0 | 0 | 10 | 8 |
| Low dosage | 0.3 | 0.03 | 10 | 8 |
| Medium dosage | 1 | 0.1 | 10 | 8 |

¹⁾Water for use in the injection was administrated.

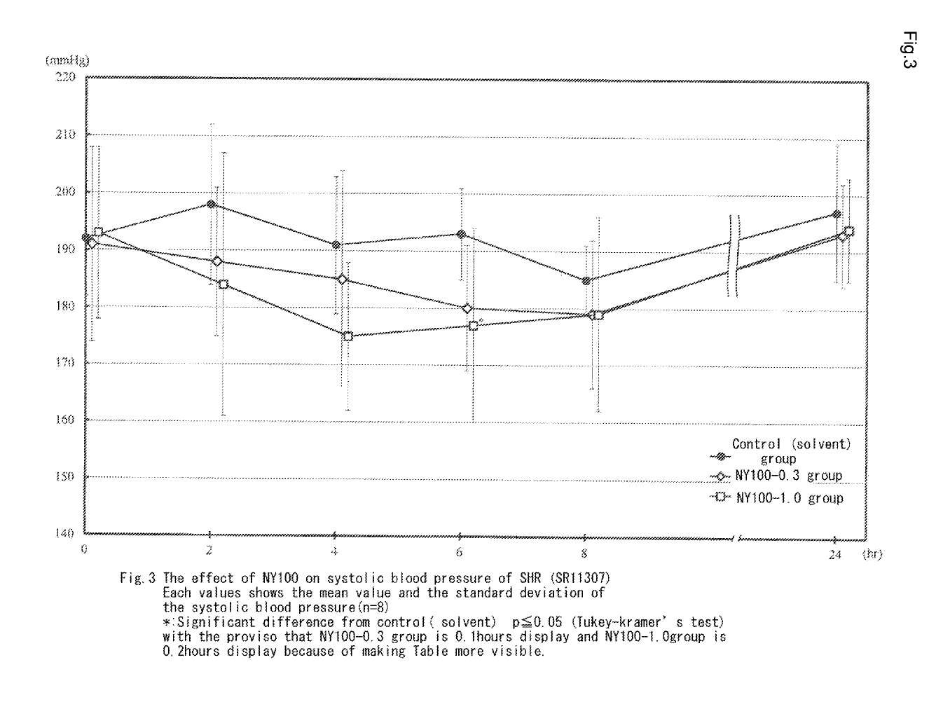

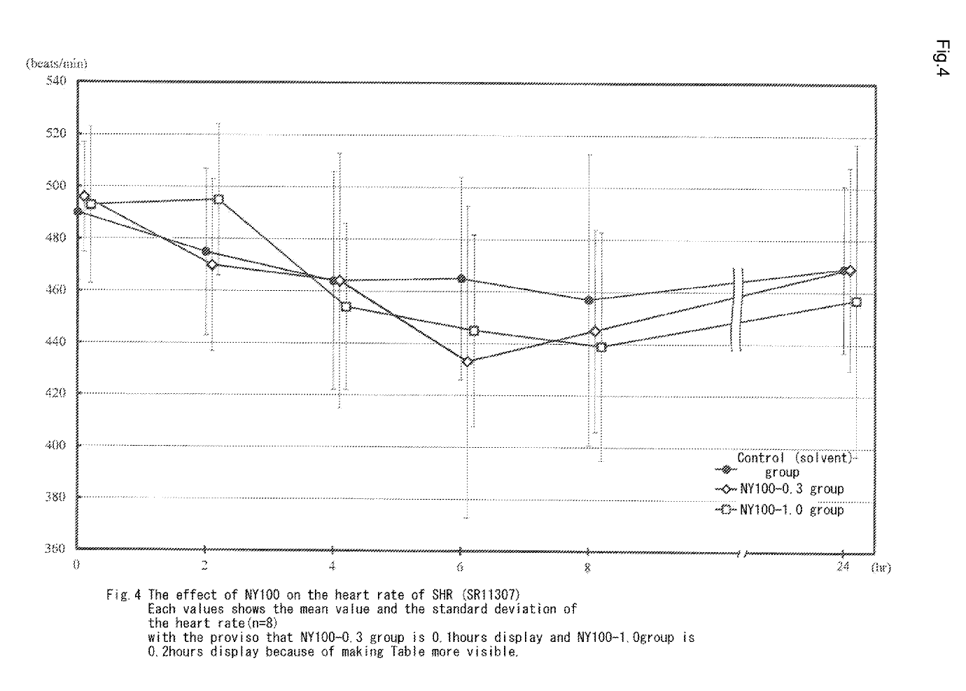

As the result, the administration of 0.3 and 1 mg/kg of KATSUOBUSHI peptide in Example 7 showed the anti-hypertensive action (FIGS. 3 and 4, and Tables 26, 27 and 28).

TABLE 26
Antihypertensive action of NY100 with spontaneously hypertensive rats.
Individual data 1
Test No.: SR11307
Blood pressure, heat rate measurement
Category of animal species and sex, SHR/Izm, male
Test group: control(solvent)

| | Body weight | Systolic blood pressure (mmHg) | | | | | |
|---|---|---|---|---|---|---|---|
| Animal | at injection | Before | Time after injection (hr) | | | | |
| No. | (g) | injection | 2 | 4 | 6 | 8 | 24 |
| 101 | 321 | 178 | 175 | 186 | 194 | 183 | 196 |
| 102 | 311 | 184 | 209 | 188 | 187 | 177 | 188 |
| 103 | 330 | 184 | 180 | 181 | 190 | 175 | 191 |
| 104 | 328 | 202 | 201 | 185 | 187 | 190 | 190 |
| 105 | 323 | 190 | 198 | 182 | 209 | 186 | 220 |
| 106 | 318 | 192 | 201 | 208 | 185 | 193 | 193 |
| 107 | 325 | 200 | 201 | 212 | 188 | 185 | 209 |
| 108 | 318 | 207 | 218 | 182 | 200 | 191 | 188 |
| Mean | 322 | 192 | 198 | 191 | 193 | 185 | 197 |
| SD | 6 | 10 | 14 | 12 | 8 | 6 | 12 |

| | Heart rate (beats/min) | | | | | |
|---|---|---|---|---|---|---|
| Animal | Before | Time after injection (hr) | | | | |
| No. | injection | 2 | 4 | 6 | 8 | 24 |
| 101 | 460 | 468 | 433 | 390 | 390 | 454 |
| 102 | 509 | 493 | 474 | 471 | 393 | 429 |
| 103 | 483 | 412 | 375 | 432 | 388 | 420 |
| 104 | 449 | 471 | 479 | 497 | 484 | 476 |
| 105 | 514 | 515 | 502 | 490 | 514 | 499 |
| 106 | 481 | 460 | 502 | 501 | 488 | 507 |
| 107 | 508 | 475 | 461 | 449 | 503 | 477 |
| 108 | 515 | 507 | 484 | 493 | 497 | 489 |
| Mean | 490 | 475 | 464 | 465 | 457 | 469 |
| SD | 26 | 32 | 42 | 39 | 56 | 32 |

TABLE 27
Antihypertensive effect of NY100 with spontaneously hypertensive rats.
Individual data 2
Test No.: SR11307
Blood pressure, heat rate measurement
Category of animal species and sex, SHR/Izm, male
Test group: NY100-1.3

| | Body weight | Systolic blood pressure (mmHg) | | | | | |
|---|---|---|---|---|---|---|---|
| Animal | at injection | Before | Time after injection (hr) | | | | |
| No. | (g) | injection | 2 | 4 | 6 | 8 | 24 |
| 201 | 314 | 164 | 182 | 159 | 186 | 165 | 208 |
| 202 | 314 | 178 | 188 | 186 | 186 | 179 | 183 |
| 203 | 321 | 194 | 187 | 164 | 164 | 160 | 184 |
| 204 | 323 | 194 | 206 | 194 | 179 | 184 | 199 |
| 205 | 310 | 184 | 183 | 179 | 174 | 199 | 197 |
| 206 | 313 | 215 | 185 | 221 | 200 | 185 | 195 |
| 207 | 331 | 186 | 169 | 189 | 175 | 188 | 185 |
| 208 | 329 | 212 | 207 | 184 | 177 | 175 | 194 |
| Mean | 319 | 191 | 188 | 185 | 180 | 179 | 193 |
| SD | 8 | 17 | 13 | 19 | 11 | 13 | 9 |

| | Heart rate (beats/min) | | | | | |
|---|---|---|---|---|---|---|
| Animal | Before | Time after injection (hr) | | | | |
| No. | injection | 2 | 4 | 6 | 8 | 24 |
| 201 | 499 | 458 | 422 | 423 | 452 | 456 |
| 202 | 531 | 485 | 515 | 522 | 447 | 496 |
| 203 | 493 | 495 | 453 | 497 | 438 | 492 |
| 204 | 465 | 400 | 383 | 352 | 405 | 385 |
| 205 | 502 | 446 | 434 | 373 | 502 | 502 |
| 206 | 513 | 490 | 494 | 403 | 382 | 453 |
| 207 | 477 | 486 | 501 | 416 | 486 | 468 |
| 208 | 484 | 496 | 513 | 477 | 447 | 500 |
| Mean | 496 | 470 | 464 | 433 | 445 | 469 |
| SD | 21 | 33 | 49 | 60 | 39 | 39 |

TABLE 28
Antihypertensive effect of NY100 with spontaneously hypertensive rats.
Individual data 3
Test No.: SR11307
Blood pressure, heat rate measurement
Category of animal species and sex, SHR/Izm, ♂
Test group: NY100-1.0

| | Body weight | Systolic blood pressure (mmHg) | | | | | |
|---|---|---|---|---|---|---|---|
| Animal | at injection | Before | Time after injection (hr) | | | | |
| No. | (g) | injection | 2 | 4 | 6 | 8 | 24 |
| 301 | 316 | 181 | 180 | 170 | 163 | 187 | 186 |
| 302 | 333 | 190 | 178 | 194 | 191 | 167 | 199 |
| 303 | 298 | 176 | 140 | 177 | 152 | 162 | 180 |
| 304 | 333 | 198 | 175 | 171 | 174 | 166 | 193 |
| 305 | 325 | 204 | 195 | 179 | 189 | 185 | 197 |
| 306 | 328 | 189 | 211 | 153 | 169 | 174 | 195 |
| 307 | 322 | 182 | 181 | 165 | 174 | 173 | 193 |
| 308 | 312 | 223 | 209 | 187 | 203 | 214 | 210 |
| Mean | 321 | 193 | 184 | 175 | 177* | 179 | 194 |
| SD | 12 | 15 | 23 | 13 | 17 | 17 | 9 |

| | Heart rate (beats/min) | | | | | |
|---|---|---|---|---|---|---|
| Animal | Before | Time after injection (hr) | | | | |
| No. | injection | 2 | 4 | 6 | 8 | 24 |
| 301 | 484 | 491 | 468 | 460 | 428 | 485 |
| 302 | 493 | 492 | 422 | 414 | 403 | 488 |
| 303 | 453 | 500 | 467 | 438 | 451 | 412 |
| 304 | 489 | 496 | 442 | 427 | 494 | 504 |
| 305 | 456 | 458 | 478 | 423 | 453 | 503 |
| 306 | 540 | 523 | 460 | 451 | 407 | 471 |
| 307 | 520 | 542 | 495 | 528 | 500 | 469 |
| 308 | 505 | 459 | 397 | 421 | 376 | 326 |
| Mean | 493 | 495 | 454 | 445 | 439 | 457 |
| SD | 30 | 29 | 32 | 37 | 44 | 60 |

*significant difference from control(solvent) p ≦ 0.05 (Tukey-kramer's test)

### Comparative Example

The enzymatic decomposition product, as the comparative example, was obtained according to the method shown in Example 1, excepted that the ultrafiltration was not conducted.

### Test Example 2

### The Advantage of the Composition Defined in the Invention Compared with Comparative Example

Regarding the ACE inhibitory activity and the decrease of the blood pressure owing to the single administration test to SHR, the comparative test was done between the permeable liquid obtained by the ultrafiltration in Example 1 and the comparative example. The result was shown in the following Table 29.

TABLE 29
The comparison about ACE inhibitory activity and the decrease of the blood pressure
owing to the single administration test to SHR between the permeable fraction in
Example 1 (the composition of the invention) and the comparative example
(the control)

| | | | | Titer |
|---|---|---|---|---|
| | | | Blood pressure | (the strength |
| | ACE inhibitory | | decreasing degree | calculated from the |
| | activity | | (mmHg) | dose which the |
| | (IC50) | | (Comparison with | blood pressure was |
| | (μg-protein/mL) | Dose (mg/kg) | Control) | decreased) |
| Enzymatic | 136.25 | 500 mg/kg | −15 | 1 |
| decomposition | | 150 mg/kg | 0 | — |
| product | | | | |
| (comparative | | | | |
| example) | | | | |
| KATSUOBUSHI | 33.20 | 1 mg/kg | −16* | 500 |
| peptide of the | | | (6 hours after | |
| invention | | | administrarion) | |
| (Example 1) | | 0.3 mg/kg | −13 | 1500 |
| | | | (6 hours after | |
| | | | administrarion) | |

*significant difference from control (solvent) p ≦ 0.05 (Tukey-kramer's test)

The comparison of the inhibitory activity values between the dipeptides comprised in KATSUOBUSHI peptide of the invention and the other dipeptides, was shown in the following Table 30.

TABLE 30
The comparison of the inhibitory activity values between the dipeptides
obtained in the invention and the other peptides

| | ACE inhibitory value IC₅₀ (μM) |
|---|---|
| Dipeptides of the invention | |
| Tryptophan-isoleucine | 33.05 |
| Leucine-tryptophan | 22.97 |
| Tryptophan-leucine | 25.14 |
| Valine-tyrosine | 11.02 |
| Tryptophan-asparagine | 580.2 |
| Valine-tryptophan | 1.60 |
| Tryptophan-tyrosine | 57.00 |
| Tryptophan-methionine | 36.48 |
| Methionine-tryptophan | 3.80 |
| Isoleucine-tryptophan | 2.00 |
| Serine-tryptophan | 22.10 |
| Asparagine-tryptophan | 25.00 |
| Glutamine-tryptophan | 32.60 |
| Glycine-tryptophan | 30.00 |
| Alanine-tryptophan | 10.00 |
| Dipeptides | |
| (not be one of the invention) | |
| Proline-glycine | 17000 |
| Isoleucine-phenylalanine | 930 |
| Valine-proline | 420 |
| Glycine-aspartic acid | 9200 |

Dipeptides comprised in the composition of the invention had, as a whole, the higher ACE inhibitory activity value than those ones not comprised in the said composition. Amongst them, valine-tryptophan, isoleucine-tryptophan and methionine-tryptophan had much higher value than the other dipeptides.

The contribution rate in KATSUOBUSHI peptide of peptides of the invention was analyzed, and the result in Table 31 was shown.

TABLE 31
The contribution rate in KATSUOBUSHI
peptide of dipeptides of the invention (%)

| | The contribution rate (%) |
|---|---|
| Valine-tryptophan | 4.031 |
| Isoleucine-tryptophan | 2.040 |
| Methionine-tryptophan | 0.343 |
| Tryptophan-tyrosine | 0.021 |
| Tryptophan-methionine | 0.054 |
| Serine-tryptophan | 0.099 |
| Asparagine-tryptophan | 0.099 |
| Glutamine-tryptophan | 0.048 |
| Glycine-tryptophan | 0.366 |
| Alanine-tryptophan | 0.672 |

The contribution rate in KATSUOBUSHI peptide was high in valine-tryptophan, isoleucine-tryptophan and alanine-tryptophan, and also, it was supported that KATSUOBUSHI peptide was purified.

1. A composition characterized by being derived from a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish, and comprising a dipeptide having an angiotensin converting enzyme inhibitory activity, wherein the dipeptide comprises at least one dipeptide selected from a group consisting of
a dipeptide composed of a tryptophan-leucine amino acid sequence,
a dipeptide composed of a leucine-tryptophan amino acid sequence,
a dipeptide composed of a tryptophan-isoleucine amino acid sequence,
a dipeptide composed of a valine-tyrosine amino acid sequence,
a dipeptide composed of a tryptophan-asparagine amino acid sequence,
a dipeptide composed of a valine-tryptophan sequence,
a dipeptide composed of a tryptophan-tyrosine sequence,
a dipeptide composed of a tryptophan-methionine sequence,
a dipeptide composed of a methionine-tryptophan sequence,
a dipeptide composed of a isoleucine-tryptophan sequence,
a dipeptide composed of a serine-tryptophan sequence,
a dipeptide composed of an asparagine-tryptophan sequence,
a dipeptide composed of a glutamine-tryptophan sequence,
a dipeptide composed of a glycine-tryptophan sequence and
a dipeptide composed of an alanine-tryptophan sequence, and/or acid addition salts thereof.

2. A composition according to claim 1, characterized in that the composition comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence and a dipeptide composed of a tryptophan-asparagine amino acid sequence.

3. A composition according to claim 1, characterized in that the composition comprises a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence and a dipeptide composed of an isoleucine-tryptophan sequence.

4. A composition according to claim 1, characterized in that the composition comprises a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

5. A composition according to claim 1, characterized in that the composition comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence, and/or acid addition salts thereof.

6. A processed food or a food for specified health use, characterized by comprising a composition according to any one of claims 1 to 5.

7. A pharmaceutical composition, characterized by comprising a composition according to any one of claims 1 to 5.

8. A pharmaceutical composition according to claim 7, wherein the pharmaceutical composition is an antihypertensive composition.

9. A method for producing a composition comprising at least one dipeptide selected from a group consisting of a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence, and/or acid addition salts thereof, characterized in that the method consists of:
1) extracting a fish meat protein of a bonito, a roughly dried bonito, a really dried bonito, a frigate mackerel, a dried frigate mackerel, a sardine, a dried sardine, a saurel, a dried saurel, a mackerel, a dried mackerel, a dried small sardine, or the other a miscellaneous dried fish with hot water,
2) grinding a water-insoluble protein remained after the hot water-extraction, and reacting, the water-insoluble protein particle obtained by dispersing the obtained ground product into water, with a protease under the optimum condition of pH5.0 to pH9.0 at a temperature of 40 to 60° C., and thereby, enzymatically hydrolyzing the water-insoluble protein, and then, terminating the enzyme reaction, and removing water-insoluble particles from the obtained hydrolysis reaction mixture including water, and thereby, obtaining aqueous solution comprising hydrophobic high molecular, hydrophilic high molecular and low molecular peptide as well as a water-soluble amino acid, and
3) loading and passing a bound fraction obtained with hydrophobic resin column method to ultrafiltration (molecular weight: 1000) to finally purify the fraction.

10. A composition, obtained by use of the method according to claim 9.

11. A composition according to claim 10, characterized in that the composition comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence and a dipeptide composed of a tryptophan-asparagine amino acid sequence.

12. A composition according to claim 10, characterized in that the composition comprises a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence and a dipeptide composed of an isoleucine-tryptophan sequence.

13. A composition according to claim 10, characterized in that the composition comprises a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

14. A composition according to claim 10, characterized in that the composition comprises a dipeptide composed of a tryptophan-leucine amino acid sequence, a dipeptide composed of a leucine-tryptophan amino acid sequence, a dipeptide composed of a tryptophan-isoleucine amino acid sequence, a dipeptide composed of a valine-tyrosine amino acid sequence, a dipeptide composed of a tryptophan-asparagine amino acid sequence, a dipeptide composed of a valine-tryptophan sequence, a dipeptide composed of a tryptophan-tyrosine sequence, a dipeptide composed of a tryptophan-methionine sequence, a dipeptide composed of a methionine-tryptophan sequence, a dipeptide composed of a isoleucine-tryptophan sequence, a dipeptide composed of a serine-tryptophan sequence, a dipeptide composed of an asparagine-tryptophan sequence, a dipeptide composed of a glutamine-tryptophan sequence, a dipeptide composed of a glycine-tryptophan sequence and a dipeptide composed of an alanine-tryptophan sequence.

15. A processed food or a food for specified health use, characterized by comprising a composition according to any one of claims 10 to 14.

16. A pharmaceutical composition, characterized by comprising a composition according to any one of claims 10 to 14.

17. A pharmaceutical composition according to claim 16, wherein the pharmaceutical composition is an antihypertensive composition.
